(19) United States
(12) Patent Application Publication
(10) Pub. No.: US 2024/0002916 A1
(43) Pub. Date: Jan. 4, 2024

(54) DIRECT LYSIS APPARATUS AND METHODS FOR MOLECULAR DIAGNOSTICS

(71) Applicant: NUCLEIN, LLC, Austin, TX (US)

(72) Inventors: Richard CROCKETT, Austin, TX (US); Alan BLAKE, Austin, TX (US); Brittan PASLOSKE, Austin, TX (US); Jeffrey SHELTON, Austin, TX (US); Michael KARBERG, Austin, TX (US); Ehren ACHEE, Austin, TX (US); James Doug WEHRLY, Austin, TX (US); Alex ILES, Austin, TX (US); John LUPHER, Austin, TX (US)

(73) Assignee: NUCLEIN, LLC, Austin, TX (US)

(21) Appl. No.: 18/038,181

(22) PCT Filed: Nov. 16, 2021
(86) PCT No.: PCT/US2021/072419

§ 371 Date: May 22, 2023

Related U.S. Application Data
(60) Provisional application No. 63/117,317, filed on Nov. 23, 2020.

(51) Int. Cl. C12Q 1/6844; B01L 3/00; B01L 7/00; C12Q 1/6806

(52) U.S. Cl. CPC C12Q 1/6844; B01L 3/502753; B01L 7/52; C12Q 1/6806; B01L 2200/16; B01L 2300/0681; B01L 2300/18

(57) ABSTRACT

This disclosure relates to direct lysis apparatus and methods for molecular diagnostics. Certain embodiments include a piston cycled from a first position proximal to a first end of a housing, to a second position proximal to a second end of the housing, and back to the first position proximal to the first end of the housing. In some embodiments, the present disclosure relates to devices, methods, and systems for molecular diagnostics that do not comprise a piston.

### CROSS-REFERENCE TO RELATED APPLICATIONS

This application claims priority to U.S. Provisional Patent Application Ser. No. 63/114,987 filed Nov. 17, 2020 and to U.S. Provisional Patent Application Ser. No. 63/117,317 filed Nov. 23, 2020, the entire contents of each of which are incorporated by reference herein.

### INCORPORATION OF SEQUENCE LISTING

The sequence listing that is contained in the file named “NCNLP0009WO_ST25.txt”, which is 17 KB (as measured in Microsoft Windows) and was created on Nov. 16, 2021, is filed herewith by electronic submission and is incorporated by reference herein.

### BACKGROUND

### A. Field

This disclosure relates to apparatus and methods for molecular diagnostics. More particularly, this disclosure relates to performing molecular diagnostics by a non-technical user via a portable or disposable device that can provide sample-to-result diagnostics.

### B. Related Art

Molecular diagnostics can provide many benefits including early detection of diseases, disorders, or other genetic health-related conditions. Many molecular diagnostic techniques are based on the detection and identification of specific nucleic acids, both deoxyribonucleic acid (DNA) and ribonucleic acid (RNA), extracted and amplified from a biological specimen (e.g. blood, saliva or other substances as disclosed herein). Accordingly, while molecular diagnostics provide many benefits, typical molecular diagnostic devices are complicated, expensive, non-portable and require additional equipment and technical expertise for sample preparation, analysis, etc.

Despite barriers associated with typical devices, molecular diagnostic tests have the potential to improve health care services, enhance patient outcomes and individualized patient care. In view of the above, there is a need for molecular diagnostics provided by a stand-alone, inexpensive, simple-to-use, sample-to-result, portable or disposable device suitable for point-of-care use.

### SUMMARY

Briefly, the present disclosure provides devices, kits, methods, and systems for molecular diagnostics, along with related devices, kits, methods, and systems for the direct sample lysis, nucleic acid amplification, and detection.

Certain aspects and elements of the embodiments disclosed herein may be combined with aspects and elements of embodiments disclosed in International Patent Application No. PCT/US2019/067537, published as Patent Publication WO 2020/132279, and U.S. Provisional Patent Application Ser. No. 62/987,185 and U.S. Provisional Patent Application Ser. No. 63/114,987, the entire contents of each of which are incorporated herein by reference.

Certain embodiments include an apparatus for performing molecular diagnostics, where the apparatus comprises: a chamber (comprising a piston and a fluid configured to amplify a nucleic acid via thermal cycling); a plurality of reservoirs; a plurality of reaction components, where each reaction component is contained in a reservoir of the plurality of reservoirs; and a control circuit configured to receive a first signal from a user interface and transmit a second signal to the array of reaction component, wherein the second signal causes the release of a reaction component from a reservoir into the chamber.

In particular embodiments the plurality of reservoirs is arranged in an array. In some examples the user interface is incorporated into the apparatus, while in other examples the user interface is incorporated into a separate device that transmits the first signal to the control circuit. In some embodiments the separate device is a smart phone, table or computer. In specific embodiments the second signal is an electric or magnetic signal. In certain aspects the reaction component from the array of reaction components is a primer set is configured to replicate a nucleic acid and identify a particular analyte. In particular embodiments the piston is configured to reciprocate in the chamber.

Some examples further comprise a light source configured to illuminate the reaction components chamber. In particular examples, the chamber comprises a first end and a second, and the apparatus comprises a first heat source proximal to the first end of the chamber. Certain examples further comprise a second heat source proximal to a second end of the chamber. Specific embodiments further comprise an actuator configured to move the piston in a cycle from a first position proximal to the first end of the chamber, to a second position proximal to the second end of the chamber, and back to the first position proximal to the first end of the chamber. In some embodiments, the piston is configured to direct the fluid to the second end of the chamber when the piston is in the first position, and the piston is configured to direct the fluid to the first end of the chamber when the piston is in the second position.

Particular examples further comprise a controller configured to control the actuator. In certain embodiments the controller is configured to control the first heat source and the second heat source; the controller is configured to control the first heat source to heat the fluid proximal to the first end of the chamber to a temperature between about 92 and 98 degrees Celsius; the controller is configured to control the second heat source to heat the fluid proximal to the second end of the chamber to a temperature between about 60 and 65 degrees Celsius; and the controller is configured to cycle the piston from the first position to the second position and back to the first position between about 30 and 50 cycles. In specific embodiments the nucleic acid is DNA or RNA. In some examples, the fluid comprises polymerase chain reaction (PCR) reagents or reverse transcriptase polymerase chain reaction (RT-PCR) reagents. Particular embodiments further comprise an illumination module configured to illuminate an analyte contained in the fluid. Specific embodiments further comprise a detection module configured to detect a response from the analyte contained in the fluid. In certain examples, the detection module detects a response from the analyte in real time during amplification.

In particular embodiments the detection module detects a response from the analyte within an amplification cycle. In some embodiments the amplification cycle has a variable length. In some examples, the apparatus weighs less than five pounds, or more particularly less than four pounds, or more particularly less than three pounds, or more particularly less than two pounds, or more particularly less than one pound. In certain examples, the apparatus has two or fewer moving parts.

In some embodiments, the apparatus is powered by a battery having a capacity not greater than 7500 milliampere hours (mAh), 6,000 milliampere hours (mAh), or more particularly not greater than 5,000 mAh, or more particularly not greater than 4,000 mAh, or more particularly not greater than 3,000 mAh. In particular examples, the apparatus may have an operating noise level of less than 30 decibels, or more particularly less than 20 decibels, or more particularly less than 15 decibels, or more particularly less than 10 decibels. In certain embodiments, the apparatus is able to perform molecular diagnostics after being subjected to a fall of up to five feet onto a rigid surface. In particular examples, apparatus according to any preceding claim wherein the chamber has a volume of less than about 500 μL, 400 μL, 250 μL, 150 μL, 100 μL, 50 microliters, or less.

Particular embodiments include a method of analyzing a biological sample, where the method comprises: obtaining an apparatus for performing molecular diagnostics and selecting a desired reaction component of a plurality of reaction components via a user interface. In specific embodiments the apparatus obtained to perform the method comprises: a chamber comprising a piston and a fluid, wherein the fluid is configured to amplify a nucleic acid via thermal cycling; a plurality of reservoirs; a plurality of reaction components, wherein each reaction component is sealed in a reservoir of the plurality of reservoirs; and a control circuit configured to receive a first signal from a user interface and transmit a second signal, wherein the second signal causes the release of a desired reaction component of the plurality of reaction components from a reservoir into the chamber; and selecting the desired reaction component of the plurality of reaction components via the user interface. In certain examples, the method further comprises reviewing molecular diagnostic test results of the apparatus.

Certain embodiments include an apparatus for nucleic acid amplification, where the apparatus comprises: a receptacle configured to receive a sample; a heater; an amplification chamber; and a reaction mix, where: the apparatus is configured to heat the reaction mix to a temperature of at least 65° C. (or in some embodiments, at least about 50° C., 55° C., 60° C., 63° C., 67° C., 70° C., 75° C., 80° C., 85° C., 90° C., 94° C., 95° C., 97° C., 98° C., 99° C., 100° C., or higher); and the apparatus is configured to combine the sample with reaction mix after the reaction mix has been heated to the temperature of at least 65° C. (or in some embodiments, at least about 50° C., 60° C., 63° C., 67° C., 70° C., 75° C., 80° C., 85° C., 90° C., 94° C., 95° C., 97° C., 98° C., 99° C., 100° C., or higher). In particular embodiments, the apparatus is configured to heat the reaction mix to a temperature of approximately 95° C. Some embodiments further comprise a diluent.

In specific embodiments the apparatus is configured to heat the diluent and the reaction mix to a temperature of at least 65° C. (or in some embodiments, at least about 50° C., 55° C., 63° C., 67° C., 70° C., 75° C., 80° C., 85° C., 90° C., 94° C., 95° C., 97° C., 98° C., 99° C., 100° C., or higher), and the apparatus is configured to combine the sample with the diluent and the reaction mix after the diluent and the reaction mix has been heated to the temperature of at least 65° C. (or in some embodiments, at least about 50° C., 55° C., 60° C., 63° C., 67° C., 70° C., 75° C., 80° C., 90° C., 94° C., 95° C., 97° C., 98° C., 99° C., 100° C., or higher). In certain embodiments the apparatus is configured to heat the diluent and the reaction mix to a temperature of approximately 95° C. Particular embodiments further comprise a prefilter. In some embodiments the prefilter is configured to filter the sample after the sample is received in the receptacle and prior to the sample being introduced into the amplification chamber. In certain embodiments the prefilter is configured to mechanically filter the sample. In specific embodiments the reaction mix comprises a polymerase enzyme. In particular embodiments the polymerase enzyme is a thermostable DNA polymerase with reverse transcriptase activity. In some embodiments the thermostable polymerase with reverse transcriptase activity is Tth polymerase. In certain embodiments the polymerase enzyme is a thermostable DNA polymerase. In particular embodiments the thermostable DNA polymerase is Taq polymerase.

In some embodiments the reaction mix contains a reducing agent. In certain embodiments the reducing agent comprises dithiothreitol (DTT). In particular embodiments the reducing agent comprises tris(2-carboxyethyl)phosphine) (TCEP). In some embodiments, the sample and the reducing reagent are combined prior to being mixed with the reaction mix. In some embodiments the apparatus is configured to cycle the sample and the reaction mix between a first temperature and a second temperature to amplify nucleic acids in the amplification chamber, wherein the first temperature is a lower temperature than the second temperature. In specific embodiments the receptacle is configured as a spittoon.

Certain embodiments include a method of nucleic acid amplification, where the method comprises: filtering a raw biological sample to provide a filtered biological sample; heating a reaction mix to a temperature of at least 65° C. (or in some embodiments, at least about 50° C., 55° C., 60° C., 63° C., 67° C., 70° C., 75° C., 80° C., 85° C., 90° C., 94° C., 95° C., 97° C., 98° C., 99° C., 100° C., or higher) to provide a heated reaction mix; and combining the filtered biological sample with the heated reaction mix. In particular embodiments the reaction mix is heated to a temperature of approximately 95° C. Some embodiments further comprise diluting the filtered biological sample prior to combining the filtered biological sample with the heated reaction mix. In specific embodiments filtering the raw biological sample comprises mechanically filtering the raw biological sample. In certain embodiments the reaction mix comprises a polymerase enzyme. In particular embodiments the polymerase enzyme comprises a thermostable DNA polymerase with reverse transcriptase activity. In some embodiments the polymerase enzyme comprises a Tth enzyme. In certain embodiments the polymerase enzyme comprises a DNA polymerase. In specific embodiments the DNA polymerase comprises a Taq enzyme. In particular embodiments the reaction mix contains a reducing agent. In some embodiments the reducing agent comprises dithiothreitol (DTT). In certain embodiments the reducing agent comprises tris(2-carboxyethyl)phosphine) (TCEP). Particular embodiments further comprise cycling the filtered sample and the reaction mix between a first temperature and a second temperature to amplify nucleic acids in the filtered sample and reaction mix, wherein the first temperature is a lower temperature than the second temperature.

Certain embodiments include an apparatus for nucleic acid amplification, the apparatus comprising: a mechanical prefilter; a diluent; a reaction mix; and an amplification chamber, where: the diluent and reaction mix are combined into a mixture and raised to a temperature of at least 65° C. (or in some embodiments, at least about 50° C., 55° C., 60° C., 63° C., 67° C., 70° C., 80° C., 85° C., 90° C., 94° C., 95° C., 97° C., 98° C., 99° C., 100° C., or higher); and a biological sample is combined with the mixture after it has reached at least 65° C. (or in some embodiments, at least about 50° C., 55° C., 60° C., 63° C., 67° C., 70° C., 75° C., 80° C., 85° C., 90° C., 94° C., 95° C., 97° C., 98° C., 99° C., 100° C., or higher). In particular embodiments the reaction mix contains a reducing agent. In some embodiments the reaction mix is lyophilized. In certain embodiments, the mechanical prefilter is optional.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

Unless defined otherwise herein, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. In addition, the various features and embodiments of the invention contain aspects which may be used in combinations and permutations not described in particular embodiments described herein, but those skilled in the relevant art will recognize and appreciate that such combinations or permutations are within the spirit and scope of the invention. Therefore, many modifications may be made to adapt a particular situation or material to the essential scope and spirit of the invention. The disclosures of all patents, patent applications and publications cited herein are hereby incorporated herein by reference in their entireties, to the extent that they are consistent with the present disclosure set forth herein.

Reference throughout this specification to “one embodiment”, “an embodiment”, “some embodiments”, “particular embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention. Whether specifically noted as non-limiting examples or not, language describing examples, including “such as”, “including”, “other instances”, “merely exemplary”, “for instance”, “for example”, “etc.”, “e.g.”, “as well as”, “the like”, and similar terms are understood to be non-limiting.

Titles and headings of sections of this disclosure are for convenience only and shall not affect the scope or interpretation of any aspect of this disclosure.

It is understood that variations of components and/or parameters discussed in relation to one embodiment described herein can be incorporated into other embodiments described herein. In non-limiting examples, ranges and related gradations for different temperatures, pressures, time, number of cycles, ratios, volumes, dimensions, current, voltage, fluorescence, brightness, and/or distances, etc. discussed in relation to one embodiment can also be incorporated into other embodiments disclosed herein. In addition, different configurations of components, including non-limiting examples such as pistons, chambers, cylinders, probes, sensors, power sources, detectors, lysis, and/or reagents, etc. discussed in relation to one embodiment can also be incorporated into other embodiments disclosed herein.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

At least a portion of embodiments discussed herein can be implemented using a computer communicatively coupled to a network (for example, the Internet), another computer, or in a standalone computer, or as an Internet of Things (IoT) device (i.e., a smart device which may function on [i.e., as part of] the Internet of Things). As is known to those skilled in the art, a suitable computer can optionally include a processor or central processing unit (“CPU”), at least one read-only memory (“ROM”), at least one random access memory or volatile memory store (“RAM”), at least one non-volatile memory store, for example flash memory or a hard drive (“HD”), and one or more input/output (“I/O”) device(s). The I/O devices can include a keyboard, monitor, LCD screen, LEDs, input buttons or other actuators, printer, internal sensors related to the physical state, position, activity, history, magnetic field, or other aspects of the device, electronic pointing device (for example, mouse, trackball, stylist, touch pad, etc.), or the like.

ROM, RAM, and HD are computer memories for storing computer-executable instructions executable by the CPU or capable of being complied or interpreted to be executable by the CPU. Suitable computer-executable instructions may reside on a computer readable medium (e.g., ROM, RAM, and/or HD), hardware circuitry or the like, or any combination thereof. Within this disclosure, the term “computer readable medium” or is not limited to ROM, RAM, and HD and can include any type of data storage medium that can be read by a processor. For example, a computer-readable medium may refer to a data cartridge, a data backup magnetic tape, a floppy diskette, a flash memory module or drive, an optical data storage drive, a CD-ROM, ROM, RAM, HD, or the like. Software implementing some embodiments disclosed herein can include computer-executable instructions that may reside on a non-transitory computer readable medium (for example, a disk, CD-ROM, a memory, etc.). Alternatively, the computer-executable instructions may be stored as software code components on a direct access storage device array, magnetic tape, floppy diskette, optical storage device, or other appropriate computer-readable medium or storage device.

Any suitable programming language (e.g. C, Assembler, Perl, Python, Java, PHP, Ruby, Swift, Cobol) can be used to implement the routines, methods or programs of embodiments of the invention described herein, including the custom script. Other software/hardware/network architectures may be used. For example, the software tools and the custom script may be implemented on one computer or shared/distributed among two or more computers in or across a network. Communications between computers implementing embodiments can be accomplished using any electronic, optical, radio frequency signals, or other suitable methods and tools of communication in compliance with known network protocols. Additionally, any signal arrows in the drawings/figures should be considered only as exemplary, and not limiting, unless otherwise specifically noted. Based on the disclosure and teachings provided herein, a person of ordinary skill in the art will appreciate other ways and/or methods to implement the invention.

Any embodiment of any of the present methods, composition, kit, and systems may consist of or consist essentially of—rather than comprise/include/contain/have—the described steps and/or features. Thus, in any of the claims, the term “consisting of” or “consisting essentially of” may be substituted for any of the open-ended linking verbs recited above, in order to change the scope of a given claim from what it would otherwise be using the open-ended linking verb.

The use of the term “or” in the claims is used to mean “and/or” unless explicitly indicated to refer to alternatives only or the alternatives are mutually exclusive, although the disclosure supports a definition that refers to only alternatives and “and/or.”

As used herein, the terms “about”, “substantially” or “approximately” is used to indicate that a value includes the standard deviation of error for the device or method being employed to determine the value as well as an allowance of plus or minus ten-percent (10%).

As used herein, the terms “proximal to”, “near”, “adjacent to”, “disposed near” are used to indicate that two entities are related spatially and in proximity.

As used herein, the terms “amplify”, “amplification”, “replicate”, “replication”, and related terms when used in the context of nucleic acids, means to increase the amount or concentration of an identical or similar nucleic acid or nucleic acid sequence or related compound.

As used herein, “PCR” means polymerase chain reaction, but may also apply to other methods of nucleic acid amplification, for example, the various forms of isothermal amplification.

As used herein, “Tth” or “Tth DNA polymerase” refers to all thermostable DNA polymerases with reverse transcriptase activities, including recombinant and modified versions, regardless of whether they are originally derived from Thermus thermophilus.

As used herein, “bases”, “basepairs”, “bp” or the like when used in the context of nucleic acids, refers to one or more nucleotide bases, whether DNA, RNA, or XNA, and whether in single-stranded, double-stranded, triple-stranded, or other formats.

As used herein, “controlling” a temperature is used to indicate the active regulation of an element or elements in an attempt to maintain an approximate temperature range for some portion of the device, or the design elements which permit by passive or regulated loss of heat to the environment or other elements such heat loss as to achieve or attempt to achieve an approximate temperature range.

As used herein, “opposite” is used to indicate in a direction on average that is different to, orthogonal to, or opposing the general net vector of the first direction.

As used herein, when referring to movement of, or action on, a fluid, it is understood that this includes movement of, or action on, a fraction or subset of the fluid.

Following long-standing patent law, the words “a” and “an,” when used in conjunction with the word “comprising” in the claims or specification, denotes one or more, unless specifically noted.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, including the claims that follow, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein, the meaning of “in” includes “in” and “on” unless the context clearly dictates otherwise.

As used herein, “patient” or “subject” includes (whether living or not) mammalian organisms, such as human and non-human mammals, for example, but not limited to, rodents, mice, rats, non-human primates, companion animals such as dogs and cats as well as livestock, e.g., sheep, cow, horse, etc., as well as non-mammalian animals such as birds, reptiles, fishes, insects, crustaceans, arachnids, echinoderms, worms, mollusks, sponges, and other nucleic acid-bearing life such as Bacteria, viruses, Fungi, Protozoa, Archaea, Chromista and other plant life. Therefore, for example, although the described embodiments illustrate use of the present methods on humans, those of skill in the art would readily recognize that these methods and compositions could also be applied to veterinary medicine as well as on other animals and other types of organisms described herein.

As used herein, the terms “piston”, “pistons” and related terms include a mass that moves from a first position to a second position (and potentially back to the first position) in a substantially linear, or by means of mechanical linkage effects motion along such a path. The mass may be a displacer piston, which is a piston that is used to move fluid from one location to another. The mass, in certain embodiments, may be a solid, unitary component, or may comprise multiple components including for example, multiple discs, polyhedrons or coated granules which may be of similar or different shapes.

As used herein, the term “dilution fluid” and related terms include any fluid that is mixed with another substance (e.g. fluid or solid) to dilute the concentration of the substance.

As used herein, “room temperature” is used to indicate a temperature of about ten (10) to about thirty (30) degrees Celsius.

As used herein, “stable”, “stability”, or “stabilizer” in the context of reagents indicates that the critical performance attributes are not known to change by more than about 25% between the two time points.

As used herein, “metal” or “metallic” shall include alloys or mixtures of metals, including mixtures of metal incorporating non-metallic elements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a perspective view of an apparatus according to a first exemplary embodiment of the present disclosure.

FIG. 2 is a top view of the embodiment of FIG. 1.

FIG. 3 is a first elevation view of a partial assembly of the embodiment of FIG. 1.

FIG. 4 is a second elevation view of a partial assembly of the embodiment of FIG. 1.

FIG. 5 includes a third and fourth elevation view of a partial assembly of the embodiment of FIG. 1.

FIG. 6 includes a fifth and sixth elevation view of a partial assembly of the embodiment of FIG. 1

FIG. 7 is a exploded view of a partial assembly the embodiment of FIG. 1.

FIG. 8 is an elevation view of a partial assembly the embodiment of FIG. 1.

FIG. 9 is a exploded view of a partial assembly the embodiment of FIG. 1.

FIG. 10 is a schematic and graphical overview of an optical system of the embodiment of FIG. 1.

FIG. 11 is a perspective view of an apparatus according to a first exemplary embodiment of the present disclosure.

FIG. 12 is a perspective view of an apparatus according to a first exemplary embodiment of the present disclosure with a receptacle attached.

FIG. 13 is a exploded view of a partial assembly the embodiment of FIG. 11.

### DETAILED DESCRIPTION OF THE INVENTION

Exemplary embodiments of the present disclosure include apparatus and methods for the detection of nucleic acids and performing molecular diagnostics. Particular embodiments are discussed below with reference to the drawings included in the figures. For purposes of clarity, each element referred to in the discussion below of the figures may not be labeled with a reference number in each figure.

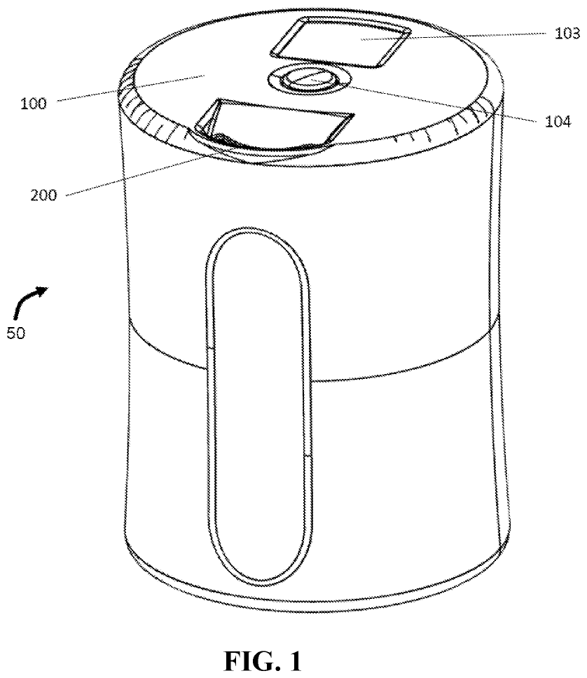

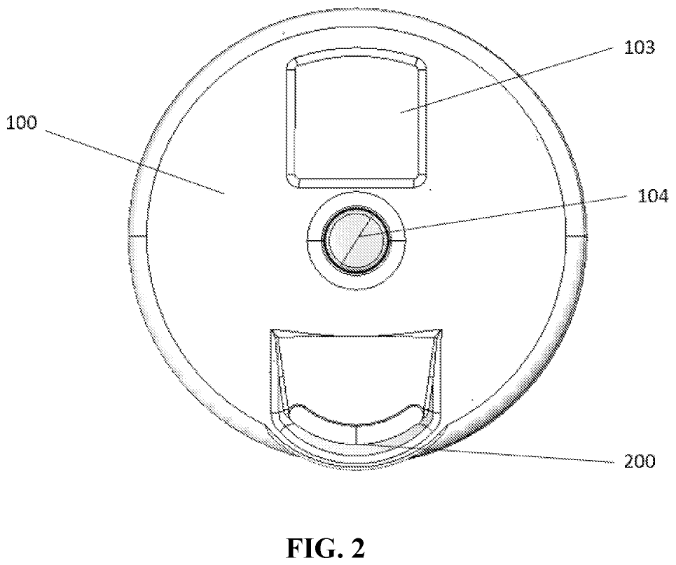

Referring initially to FIGS. 1-2, an apparatus 50 for performing molecular diagnostics is shown in perspective view. As discussed further below, a user may provide a sample into sample preparation module 200 and depress momentary button 104 to activate apparatus 50 and begin the molecular diagnostic process. In other embodiments, apparatus 50 may automatically activated by introduction of the sample into sample preparation module 200.

Prefiltering

Certain embodiments comprise an apparatus or kit for processing biological samples, where the apparatus is configured to accept a biological sample directly from the user. In some embodiments, the sample is provided by contact of the subject's body with the apparatus. In particular embodiments, this contact with the apparatus incorporates the use of a membrane, seal, or coating. In other embodiments, the user has direct contact with the housing or intake port of the apparatus. In still other embodiments, the sample is provided with the use of a pipette, transfer device, or other intermediate step that will be apparent to the practitioner.

In particular embodiments, the biological sample is saliva. In other embodiments, the sample is mucus, tears, hair, nails, hair follicle, sputum, phlegm, a buccal, nasal or nasopharyngeal swab, lacrimal fluid, rheum, blood, whole blood, plasma, serum, urine, urethral fluid, smegma, semen, vaginal secretions, breast milk, colostrum, ear wax, sebum, wound sample, pus, skin scraping, tumor, cyst, feces, cerebrospinal fluid, pericardial fluid, lymph fluid, synovial fluid, bile, gastric fluid, chyme, chyle, amniotic fluid, lochia, placenta, vitreous body, aqueous humor, material from plants, animal, bacterial, viral, fungal, archaebacteria, insects, Chromista, Protozoa, and other nucleic acid-bearing life, as well as, environmental sources such as water, air, or soil, sewage, waste, and other sources that will be apparent to the practitioner.

Some embodiments encompass an apparatus with a port for liquid biological samples. In particular embodiments, the port comprises a funnel or sloped surface to guide the liquid sample. In certain particular embodiments, the angle of approach of the guide is less than or equal to about 20°. In other embodiments, the angle is greater than about 20°, 25°, 35°, 45°, or greater. In particular embodiments, the guide relies in whole or in part on adhesion to guide the liquid sample. In certain embodiments, the guide comprises surface textures or chemical treatments to modify the relative surface energies of the guide material and the liquid sample to effect changes in the viscosity, surface tension, cohesion, or adhesion. In particular embodiments, the liquid may be channeled, or interact with microstructures. In other embodiments, surfactants or other chemicals may be deposited differentially on portions of the guide to alter the flow of the liquid sample.

In certain embodiments, the port comprises a prefilter. As used herein, the term “prefilter” refers to any filter that filters a sample prior to the sample being mixed with other reaction components, including for example, agents, buffers or other components used in nucleic acid amplification. In certain embodiments, the apparatus does not comprise a prefilter. In particular embodiments, the prefilter may be a net, sieve, porous structure, frit, screen or mesh. In some embodiments, fluid may pass through the prefilter by action of gravity. In certain embodiments, passage of the sample may be moved by vibration, for example acoustic or mechanical vibration. In other embodiments, differential pressure (including suction) may move some or all of the fluid through the prefilter. In some embodiments, the prefilter may be made of a polymerized material, such as nylon, lace, terylene net cloth, or various plastics that will be apparent to the practitioner, non-limiting examples of which are LDPE, HDPE, polypropylene (monopolymer or copolymer), polystyrene, acrylic, PTFE, polymethylpentene, PVC, polycarbonate, PFA, Delrin (acetal), PCTFE (Kel-F/Neoflon), polysulfone. In some embodiments the prefilter is made of glassified material such as borosilicate glass or other glasses that will be apparent to the practitioner. In other embodiments, the prefilter may be made of a metal, such as aluminum, steel, brass, titanium or other metals or alloys which may be formed by machining, extrusion, casting, laser or water laser cutting, 3D printing, electrical discharge machining (EDM) or other techniques that will be apparent to the practitioner.

In particular embodiments, the prefilter is a mesh with apertures of a particular size which enables the passage of liquid biological sample but tends to disproportionately exclude thicker or more viscous components of the liquid biological sample. In some embodiments, the apertures may contain between 200 and 400 apertures per square inch. In other embodiments, the apertures may be less than about 200 apertures per square inch, or less than about 175, 150, 125, 100, 75, or 50 apertures per square inch. In particular embodiments, the prefilter may incorporate pore sizes of a diameter about 0.1 μm, 0.2 μm, 0.45 μm, 1 μm, 5 μm, 10 μm, or greater.

In specific embodiments, for example where a prefilter is not used, inhibition by residual biological sample which has been incompletely lysed and persists on the solid extraction material, or filter, may inhibit amplification reactions. Biological samples may contain nucleases or other inhibitors and when present on the filter may co-elute with the desired nucleic acids. In particular embodiments, this co-elution may cause variability among samples related to the fraction of the undesired co-eluting material. In some aspects, the size of the pore may affect the binding of particles of interest within the liquid sample, for example viral particles. In certain aspects, the binding of particles of interest may be modulated by excluding particles (for example, residual food) or liquid fractions (for example, mucin). In particular embodiments, the use of a prefilter reduces the amount of undesired co-eluting material by about 10%, 25%, 50%, 75%, 85%, 90%, 95%, or more. In certain embodiments, this action may reduce the variability of the threshold of amplification (Ct) by 0.5 Ct, 1 Ct, 2 Ct, 3 Ct, 4 Ct, 5 Ct, 6 Ct, 7 Ct, 8 Ct or more.

In some embodiments, the number of apertures per square inch may be greater than about 400. In particular embodiments, the prefilter is flexible. In other embodiments, the prefilter is rigid, or semi-rigid. In certain embodiments, the apertures may be hexagonal, triangular, tetrahedral, circular, or other conformations that would be apparent to the practitioner. In some embodiments, the prefilter material is coated or treated to change its properties such as hydrophobicity and hydrophilicity, or swelling when wet. In particular embodiments, the rate at which liquid passes while under gravity is less than about 200 μL per minute. In other embodiments, the rate is greater than or equal to about 200 μL per minute, or about 250 μL, 300 μL, 500 μL, 1 mL, 5 mL, 10 mL, 25 mL, 50 mL, 75 mL, or 100 mL per minute or greater.

Nucleic Acid Extraction

Certain embodiments comprise a lysis step, where the biological sample undergoes treatment with mechanical or chemical processes to make the nucleic acids contained within the biological sample more accessible or amenable to amplification chemistries. In some embodiments, the lysis is a chemical lysis. In particular embodiments, the chemical lysis uses chaotropic salts to lyse biological material, denature proteins, or inactivate or partially inactive nucleases.

In some embodiments, the chaotropic salts are guanidine thiocyanate (guanidinium isothiocyanate or GuSCN) or guanidinium hydrochloride. In other embodiments, the chaotropic agent may be n-butanol, ethanol, lithium perchlorate, lithium acetate, magnesium chloride, phenol, 2-propanol, sodium dodecyl sulfate, thiourea, urea, sodium iodide, or other chemicals that will be apparent to the practitioner.

In certain embodiments, the lysis method comprises a solid phase extraction such as a silica substrate, for example in the form of a column, filter, or beads, including magnetic beads. In some embodiments, the solid phase extraction may be silicon oxide, aluminum silicate, activated silica, a compound which incorporates an amine group, or other such materials as will be well understood by the practitioner to aid in the isolation of nucleic acids, including RNA and DNA. In particular aspects, the lysis comprises a lysis buffer, zero or more wash steps, and an elution step. In some aspects, the lysis buffer comprises a combination lysis solution with both chaotropes and an alcohol. In particular aspects, the combination lysis solution has about 2M chaotropes, for example guanidine thiocyanate (GuSCN) and about 50% alcohol, for example ethanol.

Typically, chaotropic lysis techniques utilize a lysate incubation, for example for several minutes, followed by the addition of an alcohol prior to solid phase extraction, followed by alcohol washes (see Ali, N. et al., Current Nucleic Acid Extraction Methods and Their Implications to Point-of-Care Diagnostics., 2017, incorporated herein by reference). However, in some embodiments, it is advantageous to combine the chaotrope and alcohol into a single combination lysis solution. This simplifies sample preparation, and offers advantages to storage conditions and reliability. Typical chaotrope-based lysis includes an incubation step of at least 1 minute, followed by a series of washes (for example, see https://patents.google.com/patent/U.S. Pat. No. 7,767,804B2/en, incorporated herein by reference). In some embodiments, the use of a combination lysis solution allows the rapid and effective lysis of biological samples, for example, RNA viral samples, in less than about one minute, or less than 45 seconds, 30 seconds, 25 seconds, 20 seconds, 10 seconds, 5 seconds or 1 second.

In particular embodiments, selection of the chaotrope and the alcohol are performed such that lysis is achieved in a combination lysis solution where the precipitation temperature of the solution and the freezing point of the solution is below about 4° C., 0° C., −5° C., −10° C., −15° C., −20° C., or lower. A combination lysis solution of about 2M GuSCN and 50% ethanol is high enough concentration of chaotrope to be sufficiently effective at lysing and denaturing the biological sample, but low enough chaotrope concentration to prevent undesirable precipitation, while the ethanol concentration is high enough to lower the freezing temperature effectively. Although the freezing point of ethanol, isopropanol, and propanol are all below −20° C. at 50% concentration, in the combination lysis solution, with ethanol the precipitation or partial freezing that may occur at lower temperatures may be reversible below about 30° C., 25° C., 20° C., 10° C., 4° C., 0° C., or lower. In other embodiments, alternative alcohols in combination with chaotrope concentrations above about 2M may display irreversible precipitation without heating to above about 30° C., 40° C., 50° C., 60° C., 70° C., 80° C., 90° C. or higher. In some embodiments, formulations may precipitate at colder temperatures, for example below about −20° C., or under some conditions, below about 4° C., 0° C., −5° C., −10° C., or −15° C., but will spontaneously return to solution once returned to an ambient temperature, for example above the mentioned temperatures, or at higher temperatures such as about 10° C., 18° C., 20° C., 22° C., 25° C., or higher. In some embodiments, the return to solution may be accelerated by motion, including for example, vortex, vibratory, acoustical, mixing, Brownian, shaking, inversion, turbulence, or other motions internal to the solution or external to it.

In certain embodiments, the chaotrope concentration may be about 1.8M, 1.5M, 1.3M, 1.2M, 1M, or lower. In other embodiments, the chaotrope concentration may be increased to above about 2M, 2.2M, 2.5M, 3M, 3.5M, 4M, or higher. In certain embodiments, the molarity of the chaotrope can affect the length of the nucleotides captured by the solid phase extraction. In particular embodiments, a molarity of about 2M or higher is sufficient to capture a majority of nucleotides of at least about 20 base pairs (bp). In other embodiments, a majority of the nucleotides of at least about 25, 30, 50, 70, 100 150, 300, 500, or more base pairs may be captured by the solid phase extraction.

In some embodiments, the alcohol is ethanol. In other embodiments, the alcohol is 1-propanol, 2-propanol (isopropyl alcohol), methanol, 2-methyl-2-propanol (tert-butanol), isobutyl alcohol, but may principally be any organic compound with at least one functional hydroxl group bound to a saturated carbon atom. In certain embodiments, the combination lysis solution may contain a buffer, for example Tris HCl. In particular embodiments, the Tris HCl buffer may be at a concentration of about 5 mM, 10 mM, 15 mM, 10 mM, 25 mM, 50 mM, 75 mM, 100 mM, 150 mM, 200 mM, 225 mM, 250 mM, 300 mM or higher. In some embodiments, the pH may be about 6.5, 7, 7.5, 8, 8.5, or higher. In other embodiments, the buffer may be Tris, HEPES, TEA, MOPS, or a combination thereof.

In certain embodiments, the combination lysis solution may contain a detergent or surfactant, such as Triton X-100, Tween, SDS, NP-40, sarkosyl, Brij, CHAPS, octyl glucoside, or other chemicals that will be apparent to the practitioner. In certain embodiments, the combination lysis solution may contain a chelator, for example EDTA, EGTA, or other chemicals that will be apparent to the practitioner. In particular embodiments, the chelator will be EDTA at a concentration of about 0.5 mM, 1 mM, 2 mM, 4 mM, 6 mM, 8 mM, 10 mM, 12 mM, 14 mM, 16 mM, 18 mM, 20 mM, or higher.

In some embodiments, the ratio of combination lysis solution to biological sample volume may be less than about 4:1. In other embodiments, the minimum ratio may be about 5:1, 6:1, 7:1, 8:1, 9:1, 10:1 or higher. In particular aspects where the biological sample volume is unknown or variable, a ratio greater than the minimum ratio may be advantageous. The ratio may exceed the minimum ratio by greater than about 10%, 25%, 50%, 75%, 100%, or more.

Control

In some aspects, the apparatus may comprise an exogenous internal positive control (IPC). In certain embodiments, the control may be heterologous. In other embodiments, the control may be homologous. In particular embodiments, the control is stable at room temperature for a period of at least 10, 21, 30, 45, 60, 90, 120, 160, 365 days or more. In particular embodiments, the control is an RNA control. In other embodiments, the control is a DNA or XNA control. In some embodiments, the nucleic acid IPC is present without modification, treatment, or encapsulation (naked). In other embodiments, the nucleic acid IPC may be resistant to degradation, for example by lyophilization or encapsulation in the protein coat of a bacteriophage, for example, MS2 or Armored RNA®. In certain embodiments, stabilizers such as BSA (for example about 10 mg/mL BSA), RNAlater® or StabilZyme® may be used, optionally in conjunction with MS2 encapsulation. In particular embodiments, the concentration of MS2 in stabilizer is about 1%, 3%, 5%, 10%, 15% or 20%. In other particular embodiments the control is contained in a storage compartment such that the volume of the control and stabilizer constitutes less than about 10%, 7%, 5%, 3%, 1% or lower of the total volume of the compartment. In certain embodiments, the IPC may be stored in liquid or lyophilized format, included in the lysis buffer, or included in the elution buffer.

Filter

In certain embodiments, the lysate is passed through, or in contact with, a solid phase extraction material. In some embodiments, the solid phase extraction material may be centrifuged, or compressed, flushed with fluid (either gas or liquid) or moved by centripetal, centrifugal, vibratory, acoustic, or other forces to remove some or all of the chaotropes. In some embodiments, no alcohol wash is performed after lysis and before elution. In certain embodiments, forced hair, heat, electric charge, or vacuum may be used to remove the combination lysis solution. In particular embodiments, the chaotrope concentration in the final eluate should be below about 2%, 1.5%, 1.25%, 1%, 0.75%, 0.5%, or lower to avoid material inhibition of the subsequent amplification reaction, for example for PCR, including RT-PCR. In other embodiments which use alternate amplification chemistries (including, for example, isothermal amplification, Tth, or other thermocycling variants), the final concentration of the chaotrope may be below about 10%, 7%, 5%, 3%, 2%, 1%, 0.75%, 0.5% or lower.

In other embodiments, the solid phase extraction material is washed with a solution containing and alcohol. In particular embodiments, the alcohol is ethanol. In some embodiments, the ethanol is at least about 80%, 85%, 90%, 95%, 99%, or 100% v/v. In some embodiments, the ethanol wash solution contains additional stabilizers, including detergents, and in certain embodiments has at least about 60%, 65%, 70%, 75%, 80% or more ethanol v/v. In other embodiments the alcohol fraction is a mix of two or more alcohols.

In some embodiments, performance improvements may be achieved, for example reducing the residual alcohol concentration or increasing the effective nucleic acid capture functionality of the filter, by incorporation of a binding or compression ring which tends to minimize the passage of lysate or wash around or bypassing the filter as well as decreasing the effective liquid storage volume of the filter. In some embodiments, a similar effect may be achieved by use of a laser cutting device to form the filter components. The power of the laser may be adjusted such that in addition to cutting the filter to desired diameter (as discussed elsewhere herein), it may cause the melting or fusion of a periphery of the filter material. In other embodiments, the filters may be cut or formed using different processes, for example, die cutting or other mechanical processes that will be familiar to the practitioner, and the fusion of the filter periphery may be done in a separate process. In particular embodiments, this melt zone may be less than about 1 mm, 0.5 mm, 0.25 mm, 0.1 mm or less. In certain embodiments, the capacity of the filter, for example for lysate or residual alcohol, is proportional to the volume and mass of filter and the fraction of the filter accessible to liquids by either direct contact, capillary flow, or adhesion. In particular embodiments, it is advantageous to increase the accessible surface area per unit volume of the filter assembly to improve performance, for example to decrease the likelihood of clogging or obstructing the filter.

In specific embodiments, an advantageous conformation of the filter may be achieved by adjusting the relative surface area and thickness of the filter or the ratio between volume and the anticipated biological sample size. In certain embodiments, the ratio of the diameter of the filter to the thickness should be about 7.5 to 1. In other embodiments, the ratio is about 2:1, 3:1, 4:1, 5:1, 6:1, 7:1, 8:1, 9:1, 10:1 or higher. In particular embodiments, this ratio is modified to reflect the effect of pore size in the filter. In specific embodiments, the aforementioned ratios may be decreased by a factor for each increase of average pore size above 1 μm by decreasing the ratio by a factor of about 10-20% for each increase of average pore size of 0.25 μm or more. In other embodiments, the ratio decrease factor may be about 0%, 3%, 5% or more. In certain embodiments, the volume of the biological sample may be in a ratio of less than about 1.5:1 to the volume of the accessible filter volume. In particular embodiments, that ratio may be less than about 2:1, 3:1, 4:1, 5:1, 7:1, 10:1 or more.

In some embodiments, the solid phase extraction material is a glass fiber filter. In particular embodiments, the filter has a diameter of about 3 mm, 5 mm, 7 mm, 9 mm, 10 mm, 12 mm, 14 mm, or more. In certain embodiments, the filter has a thickness of about 0.3 mm, mm, 0.7 mm, 1 mm, 1.3 mm, 1.5 mm, 1.7 mm, 2 mm or greater. In some embodiments, the filter has a pore size of about 0.2 μm, 0.25 μm, 0.3 μm, 0.4 μm, 0.5 μm, 1 μm, 1.5 μm, 2 μm, 3 μm, 5 μm, or greater. In some embodiments, multiple filters may be used in series or in parallel. In specific embodiments, two or more filters of differing pore size may be used in series. In particular embodiments, the filter is hydrophilic. In some embodiments, the filter comprises a binder resin. In some embodiments the filter does not contain a resin binder. In certain embodiments, including those that lack a resin binder, it may be advantageous to include a support structure to mitigate shearing or perforation of the filter. The support structure can comprise perforations, for example slits or slots, curved, linear, or curvilinear apertures, holes which may be varied in size and shape, and other formats that will be apparent to the practitioner. In specific embodiments, the perforations comprise greater than about 50% of the area of the filter. In other embodiments, the perforations comprise great than about 1%, 5%, 10%, 25%, 45%, 55%, 75%, 95%, 99% or more of the area of the filter.

Pre-Elution

In certain embodiments, for example in embodiments where the apparatus does not use centripetal or centrifugal force to remove the alcohol wash, it is advantageous to minimize the residual alcohol in the filter. In particular embodiments, the residual alcohol in the filter should constitute no greater than about 5.5% of the final amplification reaction. In other embodiments, the residual alcohol should be no more than about 1%, 2%, 3%, 4%, 5%, 6%, 7%, 8%, 9%, 10% or more of the reaction. In particular embodiments, the final concentration of alcohol in an RT-PCR reaction to be no greater than about 3%, 4%, or 5%. In certain embodiments, the permissible concentration of alcohol in a reaction may be modulated by the concentration of salt present in the reaction, for example the residual chaotropic salts. In particular embodiments, the permissible alcohol may be determined by decreasing the ethanol concentration from about 5% by about 1% for each 10 mM of GuSCN present in the reaction above about 25 mM.

In some embodiments, and as described elsewhere herein, the percentage of alcohol in the alcohol may be adjusted to decrease the fraction of pure alcohol present in the wash. In other embodiments, it may be advantageous to incorporate heat or airflow or the combination of the two to increase the evaporation rate of the alcohol present in the filter. In these cases, increasing the alcohol concentration of the wash, although initially increasing the potential final alcohol concentration of the amplification reaction may be advantageous as it may increase the rate of evaporation of the alcohol when compared with a solution containing both an alcohol and aqueous component. In some embodiments, using an alcohol with a boiling point closer to the operating temperature is advantageous. For example, methanol, ethanol, 1-propanol, 2-propanol (isopropanol), 2-methyl-2-propanol, and other species that have boiling points significantly lower than that of water.

In particular embodiments where heat or airflow are used to accelerate the evaporation of the alcohol, where the wash contains an aqueous portion and the alcohol forms an azeotrope, it may be advantageous to decrease the alcohol fraction to below the azeotrope inflection (for minimum boiling azeotrope mixtures) and above the inflection point (for maximum boiling azeotrope mixtures). In certain embodiments, the incorporation of absorbent material, for example in connection with an air purge, may be useful to trap or remove wash.

In particular embodiments, the alcohol wash may be followed by, or performed in conjunction with a non-ionic, amplification-compatible wash, for example an oil wash. In some embodiments, the oil is a mineral oil, for example medium and low viscosity mineral hydrocarbons or other oils and waxes, as well as mixes of paraffinic and naphthenic liquid hydrocarbons. In particular embodiments, the boiling temperature of the oil will be above about 100° C., 120° C., 150° C., 175° C., 200° C., or higher. In certain embodiments, the oil may be used to partially or fully displace the alcohol from the solid phase extraction material prior to elution. This has the effect of pushing alcohol through the filter and may, for example, be directed to a waste chamber. Additionally, in some embodiments, the oil may displace alcohol which has adhered to the walls or chambers of the apparatus, as these can contribute to the final alcohol concentration of the amplification reaction as well if a common flow path is present.

In particular embodiments the oil may be a fatty alcohol, which can allow a higher degree of miscibility of the alcohol wash than the aqueous elution, allowing the oil wash to preferentially dissolve the alcohol wash while minimizing the solubility of aqueous compounds such as nucleic acids, which in some embodiments may allow longer chain fatty alcohols to be more effective than short chain fatty alcohols at preferentially absorbing alcohol while minimizing reductions in nucleic acid recovery. In some embodiments, fatty alcohols longer than about 10 carbons may have melting points that are higher than desired for certain applications where the application prefers a liquid fatty alcohol during the operating temperature range (for example 0° C. to 40° C., or room temperature ranges). In these cases, a heater may be employed in certain embodiments to warm the fatty alcohol prior to use as an oil wash, for example above or near its melting point.

In certain embodiments, the fatty alcohol may be tert-Butyl alcohol, tert-Amyl alcohol, 3-Methyl-3-pentanol, 2-Methyl-2-heptanol, 1-Heptanol (enanthic alcohol), 1-Octanol (capryl alcohol), Pelargonic alcohol (1-nonanol), 1-Decanol (decyl alcohol, capric alcohol), Undecyl alcohol (1-undecanol, undecanol, Hendecanol), Lauryl alcohol (dodecanol, 1-dodecanol), Tridecyl alcohol (1-tridecanol, tridecanol, isotridecanol), Myristyl alcohol (1-tetradecanol), Pentadecyl alcohol (1-pentadecanol, pentadecanol), Cetyl alcohol (1-hexadecanol), Palmitoleyl alcohol (cis-9-hexadecen-1-ol), Heptadecyl alcohol (1-n-heptadecanol, heptadecanol), Stearyl alcohol (1-octadecanol), Oleyl alcohol (1-octadecenol), Nonadecyl alcohol (1-nonadecanol), Arachidyl alcohol (1-eicosanol), Heneicosyl alcohol (1-heneicosanol), Behenyl alcohol (1-docosanol), Erucyl alcohol (cis-13-docosen-1-ol), Lignoceryl alcohol (1-tetracosanol), Ceryl alcohol (1-hexacosanol), 1-Heptacosanol, Montanyl alcohol, cluytyl alcohol, or 1-octacosanol, 1-Nonacosanol, Myricyl alcohol, melissyl alcohol, or 1-tri acontanol, 1-Dotri ac ontanol (Lacceryl alcohol), Geddyl alcohol (1-tetratriacontanol), or other fatty alcohols which will be apparent to the practitioner. In particular embodiments, the fatty alcohols may be saturated or unsaturated, and straight or branched. In some embodiments, when a straight fatty acid (non-branched) is used, a carbon backbone length of at least nine carbons is preferred in cases where the hydroxyl group is on the first carbon atom. In some embodiments, for example, when the hydroxyl group is not on the first carbon, or where branched (such as 2-Methyl-2-heptanol) or unsaturated (including partially unsaturated) fatty alcohols are used, the operating temperature range of the fatty alcohol is lower or more advantageous for use in a room temperature environment (for example, the miscibility may be higher with ethanol, and lower with the aqueous fraction). In certain embodiments, mixtures of various fatty alcohol may be used to achieve these desirable traits.

Elution

Certain embodiments comprise the use of an elution buffer. In certain embodiments, the elution buffer is water. In particular embodiments, the water is nuclease-free water. In some embodiments, the elution may comprise a microbiocide, such as sodium azide, ProClin, or other antimicrobials that will be apparent to the practitioner. In some embodiments, the elution fluid may contain salts such as KCl, MgCl₂, or other such reagents as support or participate in the amplification reaction. In particular embodiments, the salt concentration is below about 500 mM, 400 mM, 300 mM, 250 mM, 200 mM, or less. In some embodiments, the elution buffer may contain carbohydrates or stabilizers such as trehalose, mannitol, sucrose, sorbitol, PEG (polyethylene glycol), glycerol, or other reagents that will be apparent to the practitioner. In particular embodiments, the trehalose concentration may be about 1%, 3%, 5%, 7%, 10%, 12%, 15% or more. In some embodiments, the mannitol concentration may be about 0.1%, 0.5%, 1%, 1.5%, 2% or more. In certain embodiments, the elution may contain pH buffers. In other embodiments, the elution solution may contain glycerol, for example at about 0.1%, 0.5%, 1%, 1.25%, 1.5%, or more. In certain embodiments, oligonucleotide primers and probes may be included in the elution buffer.

In some embodiments, the present apparatus may be used for the detection of target nucleic acids, such as the detection of severe acute respiratory syndrome coronavirus 2 (SARS-CoV-2) sequences. Exemplary sequences that target the N or S proteins of SARS-CoV-2 are provided herein.

Target sequences corresponding to four different regions of the SARS-CoV-2 reference genome:

SEQ ID NO: 1-ORF1ab: NC_045512:3993-4235
TGGAAGAAACTAAGTTCCTCACAGAAAACTTGTTACTTTATATTGACATT
AATGGCAATCTTCATCCAGATTCTGCCACTCTTGTTAGTGACATTGACAT
CACTTTCTTAAAGAAAGATGCTCCATATATAGTGGGTGATGTTGTTCAAG
AGGGTGTTTTAACTGCTGTGGTTATACCTACTAAAAAGGCTGGTGGCACT
ACTGAAATGCTAGCGAAAGCTTTGAGAAAAGTGCCAACAGACA
SEQ ID NO: 2-ORF1ab: NC_045512:13227-13383
TTGGTGGTGCATCGTGTTGTCTGTACTGCCGTTGCCACATAGATCATCCA
AATCCTAAAGGATTTTGTGACTTAAAAGGTAAGTATGTACAAATACCTAC
AACTTGTGCTAATGACCCTGTGGGTTTTACACTTAAAAACACAGTCTGTA
CCGTCTG
SEQ ID NO: 3-S protein: NC_045512:24035-24324
AAAGTGACACTTGCAGATGCTGGCTTCATCAAACAATATGGTGATTGCCT
TGGTGATATTGCTGCTAGAGACCTCATTTGTGCACAAAAGTTTAACGGCC
TTACTGTTTTGCCACCTTTGCTCACAGATGAAATGATTGCTCAATACACT
TCTGCACTGTTAGCGGGTACAATCACTTCTGGTTGGACCTTTGGTGCAGG
TGCTGCATTACAAATACCATTTGCTATGCAAATGGCTTATAGGTTTAATG
GTATTGGAGTTACACAGAATGTTCTCTATGAGAACCAAAA
SEQ ID NO: 4-S protein: NC_045512:24035-24283
AAAGTGACACTTGCAGATGCTGGCTTCATCAAACAATATGGTGATTGCCT
TGGTGATATTGCTGCTAGAGACCTCATTTGTGCACAAAAGTTTAACGGCC
TTACTGTTTTGCCACCTTTGCTCACAGATGAAATGATTGCTCAATACACT
TCTGCACTGTTAGCGGGTACAATCACTTCTGGTTGGACCTTTGGTGCAGG
TGCTGCATTACAAATACCATTTGCTATGCAAATGGCTTATAGGTTTAAT
SEQ ID NO: 5-M protein: NC_045512:27047-27199
GCTTTCTTATTACAAATTGGGAGCTTCGCAGCGTGTAGCAGGTGACTCAG
GTTTTGCTGCATACAGTCGCTACAGGATTGGCAACTATAAATTAAACACA
GACCATTCCAGTAGCAGTGACAATATTGCTTTGCTTGTACAGTAAGTGAC
AAC
SEQ ID NO: 6-N protein: NC_045512:28523-28654
ATTGGCTACTACCGAAGAGCTACCAGACGAATTCGTGGTGGTGACGGTAA
AATGAAAGATCTCAGTCCAAGATGGTATTTCTACTACCTAGGAACTGGGC
CAGAAGCTGGACTTCCCTATGGTGCTAACAAA
SEQ ID NO: 7-N protein: NC_045512:29411-29514
CAAGCCTTACCGCAGAGACAGAAGAAACAGCAAACTGTGACTCTTCTTCC
TGCTGCAGATTTGGATGATTTCTCCAAACAATTGCAACAATCCATGAGCA
GTGC

In certain embodiments, target sequences may be detected by using primers and probes designed based on the above target sequences. Exemplary primers and probes are provided in Table 1.

TABLE 1
Primer and probe sequences.

| | | SEQ | |
|---|---|---|---|
| Assay | Sequence | ID NO: | Oligo |
| S1 | TTGCCACCTTTGCTCACAGA | 8 | Forward |
| | CACCAAAGGTCCAACCAGAA | 9 | Reverse |
| | ACACTTCTGCACTGTTAGCGGGTACA | 10 | Probe |
| N2 | CCGCAGAGACAGAAGAAACAG | 11 | Forward |
| | GCACTGCTCATGGATTGTTG | 12 | Reverse |
| | AACTGTGACTCTTCTTCCTGCTGCAGA | 13 | Probe |
| S-2 | GCACAAAAGTTTAACGGCCTTAC | 14 | Forward |
| | CGCTAACAGTGCAGAAGTGTATTGA | 15 | Reverse |
| | TGCCACCTTTGCTCACAGATGAAATGA | 16 | Probe |
| S-M30 | TGTCATGATGGAAAAGCACACTT | 17 | Forward |
| | ACCAGTGTGTGCCATTTGAAAC | 18 | Reverse |
| | CCTCGTGAAGGTGTCTT | 19 | Probe |
| ORF1ab- | TTGCTGCAGTCATAACAAGAGAAGT | 20 | Forward |
| 1 | TTGCTGCAGTCATAACAAGAGAAGT | 21 | Reverse |
| | TTTGCCTGGCACGATATTACGCACAA | 22 | Probe |
| ORF1ab- | GTGGTGCATCGTGTTGTCTGT | 23 | Forward |
| 2 | AGGGTCATTAGCACAAGTTGTAGGT | 24 | Reverse |
| | CTGCCGTTGCCACATAGATCATCCAA | 25 | Probe |
| ORF1ab- | TGACAAAGCTTGCCCATTGA | 26 | Forward |
| M9 | GCACGACAAAACCCACTTCTC | 27 | Reverse |
| | TGCTGCAGTCATAACA | 28 | Probe |
| ORF1ab- | GGGATCAGACATACCACCCAAA | 29 | Forward |
| M16 | TTTGCACAATGCAGAATGCA | 30 | Reverse |
| | TGTGTTAACTGTTTGGATGAC | 31 | Probe |
| ORF1ab- | CCGCGAAGAAGCTATAAGACATG | 32 | Forward |
| M22 | GACACCCCTCGACATCGAA | 33 | Reverse |
| | ACGTGCATGGATTGG | 34 | Probe |
| M-1 | CATTGCTTCTTTCAGACTGTTTGC | 35 | Forward |
| | GCACGTTGAGAAGAATGTTAGTTTCT | 36 | Reverse |
| | CGTACGCGTTCCATGTGGTCATTCA | 37 | Probe |
| M-M35 | GCTTCGCAGCGTGTAGCA | 38 | Forward |
| | TGCCAATCCTGTAGCGACTGT | 39 | Reverse |
| | TGACTCAGGTTTTGCTG | 40 | Probe |
| N-M36 | ACCGAAGAGCTACCAGACGAAT | 41 | Forward |
| | CCATCTTGGACTGAGATCTTTCATT | 42 | Reverse |
| | CGTGGTGGTGACGGTA | 43 | Probe |

In some embodiments, the probe, such as the exemplary probes above, comprises a minor groove binder on the 3′-end, the 5′-end, or both the 3′-end and the 5′-end of the nucleotide sequence. In some embodiments, the minor groove binder is located internally. The term “minor groove binder” as used herein refers to a small molecule that fits into the minor groove of double-stranded DNA, sometimes in a sequence specific manner. Generally, minor groove binders are long, flat molecules that can adopt a crescent-like shape and thus, fit snugly into the minor groove of a double helix, often displacing water. Minor groove binding molecules typically comprise several aromatic rings connected by bonds with torsional freedom, for example but not limited to, furan, benzene, or pyrrole rings.

In certain embodiments, the minor groove binder further comprises a quencher, for example but not limited to, a MGB-NFQ (Applied Biosystems). Non-limiting examples of minor groove binders include, antibiotics such as netropsin, distamycin, berenil, pentamidine and other aromatic diamidines, Hoechst 33258, SN 6999, aureolic anti-tumor drugs such as chromomycin and mithramycin, CC-1065, dihydrocyclopyrroloindole tripeptide (DPI₃), 1,2-dihydro-(3H)-pyrrolo[3,2-e]indole-7-carboxylate (CDPI₃), and related compounds and analogs. Descriptions of minor groove binders can be found in, among other places, Nucleic Acids in Chemistry and Biology, 2d ed., Blackburn and Gait, particularly in section 8.3; Kumar et al., Nucl. Acids Res. 26:831-38, 1998; Kutyavin et al., Nucl. Acids Res. 28:655-61, 2000; Turner and Denny, Curr. Drug Targets 1:1-14, 2000; Kutyavin et al., Nucl. Acids Res. 25:3718-1997; Lukhtanov et al., Bioconjug. Chem. 7:564-7, 1996; Lukhtanov et al., Bioconjug. Chem. 6: 418-26, 1995; U.S. Pat. No. 6,426,408; and PCT Published Application No. WO 03/078450. Those in the art understand that minor groove binders typically increase the Tₘ of the oligonucleotide to which they are attached, allowing such oligonucleotides to effectively hybridize at higher temperatures. Minor groove binders are commercially available from, among other sources, Applied Biosystems (Foster City, Calif) and Epoch Biosciences (Bothell, Wash.).

In some embodiments, an internal positive control (IPC) may be used to confirm DNA amplification, detect false negatives, and the presence of amplification inhibitors in a sample. Exemplary IPCs that may be used with the present methods are provided in Table 2.

TABLE 2
IPC target, primer and probe sequences.

| | Template | F, P, R |
|---|---|---|
| IPC1 | CGCTAACGGGCGATT | Forward |
| | CTATAAGAATGCACA | CGCTAACGGGCGATTCTAT |
| | TTGCGTCGATTCATA | (SEQ ID NO: 45)(Sense) |
| | AGATGTCTCGACCGC | Probe |
| | ATG | AAGAATGCACATTGCGTCGATTCA |
| | (SEQ ID NO: 44) | (SEQ ID NO: 46)(Sense) |
| | | Reverse |
| | | CATGCGGTCGAGACATCTTA |
| | | (SEQ ID NO: 47)(AntiSense) |
| IPC2 | ATCAAGGAAATGTTT | Forward |
| | CATGACCAAGCGAAA | ATCAAGGAAATGTTTCATGACC |
| | GGCCGCTCTACGGAA | (SEQ ID NO: 49)(Sense) |
| | TGGATTTACGTTACT | Probe |
| | GCCT | AAGCGAAAGGCCGCTCTACG |
| | (SEQ ID NO: 48) | (SEQ ID NO: 50)(Sense) |
| | | Reverse |
| | | AGGCAGTAACGTAAATCCATTC |
| | | (SEQ ID NO: 51)(AntiSense) |
| IPC3 | CTGCTTTGATCAACC | Forward |
| | TCCAATACCTCGTAT | CTGCTTTGATCAACCTCCAATAC |
| | CATTGTGCACCTGCC | (SEQ ID NO: 53)(Sense) |
| | GGTGACCACTCAACG | Probe |
| | ATGTGG | CTCGTATCATTGTGCACCTGCCGG |
| | (SEQ ID NO: 52) | (SEQ ID NO: 4)(Sense) |
| | | Reverse |
| | | CCACATCGTTGAGTGGTCA |
| | | (SEQ ID NO: 55)(AntiSense) |
| IPC4 | CGCATGCGCAGGGTA | Forward |
| | TATTTGGACAGTATC | CGCATGCGCAGGGTATATT |
| | GAATGGACTCTGATG | (SEQ ID NO: 57)(Sense) |
| | AACCTTTACACCGAT | Probe |
| | CTAGAAACGGG | TGGACAGTATCGAATGGACTCTGATGAAC |
| | (SEQ ID NO: 56) | (SEQ ID NO: 58)(Sense) |
| | | Reverse |
| | | CCCGTTTCTAGATCGGTGTAAAG |
| | | (SEQ ID NO: 59)(AntiSense) |
| IPC5 | GAGGCGAAGATTATC | Forward |
| | GTGTGTGCCCCGTTA | GAGGCGAAGATTATCGTGTGTG |
| | TGGTCGAGTTCGGTC | (SEQ ID NO: 61)(Sense) |
| | AGAGCGTCATTGCGA | Probe |
| | GTAGTCGTTTGC | CCCGTTATGGTCGAGTTCGGTCAG |
| | (SEQ ID NO: 60) | (SEQ ID NO: 62)(Sense) |
| | | Reverse |
| | | GCAAACGACTACTCGCAATGA |
| | | (SEQ ID NO: 63)(AntiSense) |

In some embodiments, the present methods comprise the detection of endogenous internal positive controls, such as RNaseP. Exemplary primer and probes that may be used are provided in Table 3.

TABLE 3
Primer and probes for detection of RPP30 mRNA.

| | Sequences (Forward, | SEQ |
|---|---|---|
| Assay | Probe, Reverse, 5′→3′) | ID NO: |
| 1 | GATGCAAATCTGCAAAGGAAAGA (Sense) | 64 |
| | TAAGAGGGCCATATGACGTGGCAA (Sense) | 65 |
| | CCCAAACAGCAAGCCTAGAT (AntiSense) | 66 |
| 2 | TGGCGGTGTTTGCAGATT (Sense) | 67 |
| | TTCTGACCTGAAGGCTCTGCGC (Sense) | 68 |
| | ACAACTGAATAGCCAAGGTGAG (AntiSense) | 69 |
| 3 | TGTCTCGGATCCATCTCACT (Sense) | 70 |
| | TGAGAGCAACTTCTTCAAGGGCCC (Sense) | 71 |
| | AAACTGCAACAACATCATAGAGC (AntiSense) | 72 |
| 4 | ACTTTGCCAATTGTACAGGGA (Sense) | 73 |
| | TGTCTCGGATCCATCTCACTGCAA (Sense) | 74 |
| | GCCCTTGAAGAAGTTGCTCT (AntiSense) | 75 |
| 5 | TATCTAGTGCTGCAGAAAGGC (Sense) | 76 |
| | AGGGCCATATGACGTGGCAAATCT (Sense) | 77 |
| | CTTGGCGTCACTTTCAGAGA (AntiSense) | 78 |

Sample Detection

In certain embodiments, the apparatus comprises a sensor to detect the presence of or quantity of, a biological sample. In some embodiments, the sensor may be a capacitive sensor, an inductive sensor, a resistive sensor, a light sensor, an opacity sensor, a float sensor, a chemical sensor, a temperature sensor, pressure sensor, or a time of flight sensor (for example, an ultrasonic or laser pulse).

In some embodiments, the sensor is positioned in proximity to or in communication with, the intake port. In particular embodiments, the intake port contains dry or concentrated elements which, when suspended by a biological sample, allow detection by the sensor. For example, metallic or magnetic particles may be used with inductive sensors. In certain embodiments, the particles may be kept separate from the intake port by means of a membrane or division, or by using particles which are of sufficient size to avoid transport along with the biological sample beyond a specific portion of the apparatus. For example, the particles may be bigger than about 0.01 mm, 0.1 mm, 0.3 mm, 0.5 mm, 1 mm or larger.

In certain embodiments, the presence or level of a sample may be detected by using a float sensor. In particular embodiments, the float sensor is a Hall effect sensor and the sample is detected as a differential density moves an object, for example a low density float such as a polystyrene or other foam with a density less than the biological sample, for example at least about 50%, 75%, 100%, or more less dense the sample. In particular embodiments the float contains a detectable element, for example a magnetic particle which is detectable by the float sensor, for example a Hall effect or induction sensor.

In some embodiments, the presence of a liquid biological sample may be detected by the use of a feature responsive to capillary action. The action of capillary action may be include the alteration of pressure within the apparatus, the refraction, attenuation, redirection, or amplification of light via a conductive or obstructive light path, detectable by sensor types that will be apparent to the practitioner.

In particular embodiments, the biological sample acts as a dielectric. In other embodiments, the biological sample is treated as a conductive media. In certain embodiments, the conductive nature of the biological sample is used as a sensor by calculating the resistance between various open circuits and a ground (or other differential voltage) state. The sensor may comprise multiple circuits stratified by known spatial domains such that conduction between different circuits detects the fluid level of the biological sample. In certain embodiments, the number of circuits are one, two, three, four, five, ten, or more. In some embodiments, the biological sample is liquid, for example saliva. In certain embodiments, the resistance of saliva is about 100-120 kohms. In particular embodiments, the resistance of the biological sample is less than 500 kohms, 400 kohms, 300 kohms, 250 kohms, 200 kohms, 150 kohms or lower. In certain embodiments, resistance measurements may be used to determine whether the biological sample is present, or whether a liquid sample is diluted, adulterated, or represents an altered physiological state.

In certain embodiments, where a liquid biological sample is used, bubbles (or foam) may form during fluid ingress or processing, or may be part of the natural biological sample. In particular modes of detection, most notably optical and resistive sensing, but also present in other modes such as pressure sensing, the presence of bubbles may present an obstacle to consistent and reliable detection. Bubbles may be mitigated by the use of anti-foaming or de-foaming agents (including surfactants) that will be familiar to the practitioner. Non-limiting examples include various oils (principally insoluble oils), silicones, alcohols, glycols and stearates. Unfortunately, in some embodiments, these chemicals may themselves affect the sample detection modes or they may interfere with various mechanical or chemical process in the sample preparation, amplification, or detection aspects of the apparatus.

In some embodiments, the use of mechanical aspects to mitigate the impacts of bubbles avoids some of the deleterious aspects of traditional anti-foaming or de-foaming. In certain aspects, the intake port may be patterned with asperities which tend to modulate the surface tension on bubbles, increasing the likelihood of bubble rupture. In particular aspects, the size of the asperities can be tailored to the mean size of the bubbles in the fluid, where an aspect ratio of mean bubble diameter to asperity size may be about 10:1, 7:1, 5:1, 3:1, 1:1 or lower. In particular embodiments where the bubbles traverse a lip or baffle, the ratio of mean bubble size to the length of the feature may be about 0.5:1, 1:1, 2:1, 3:1 or more. In other embodiments, bubbles may be excluded by use of a gate or grate feature. In certain embodiments, the ratio of the apertures of the gate or grate to the minimum acceptable bubble size may be less than about 2:1, 1.5:1, 1:1, or less.

In certain embodiments, bubbles may be addressed by passing them destructively over a substrate or surface. In some embodiments, heat, electrical current, ultrasonic or sound based approaches may be employed. In particular aspects, heating the sample causes expansion and adiabatic changes which create tension points and tend toward bubble rupture. In certain aspects when the target of interest in the biological sample is fragile, for example RNA, the average temperature of the fluid may be maintained at a temperature less than about 20° C., or less than about 22° C., 25° C., 30° C., 40° C., 50° C. or 65° C. to mitigate degradation of the target. Temperature elevation above these ranges may occur in certain embodiments without material degradation if the period is less than about 10 seconds, 5 seconds, 3 seconds, or 1 second, or if only a fraction of the sample is heated, for example, less than about 50%, 40%, 30%, 25%, 20%, 10%, 5%, 1% or less.

In other aspects, electrical currents may create short arcs which tend to rupture bubbles along their surfaces, vertices and junction points. In particular aspects, the current and voltage may be adjusted based on the electrical circuit arc path such that the temperature of the bubble exceeds the boiling point of the fluid, but the mean temperature of the fluid does not damage the biological sample of interest (as discussed herein). In certain embodiments, multiple pulses may be used to effect this balance, in some aspects with a “duty cycle” (or ratio of on-time to off-time) of less than about 50%, 25%, 10%, 5%, 1%, or less.

Reagent Sealing

In some embodiments, it is advantageous to contain the various reagents within the apparatus such that they are isolated from the environment. This may prevent spoilage, spillage, evaporation, absorption, drying, wetting, or glassing (for example of hygroscopic compounds). In certain embodiments, the various reagents may be contained in one or more reagent cartridges. In some embodiments, the reagents may be included in both liquid and dry or lyophilized form.

In certain embodiments, the reagents may be contained in one or more rigid or semi-rigid cartridges, including, for example polymer or metal forms. In other embodiments, the reagents may be contained in flexible or soft cartridge components. In particular embodiments, the cartridge components may be used to contain a liquid by use of a polymer (for example, a thermoplastic polymer such as polypropylene), foil (including wax backed foils), or other pouch which is sealed to isolate the reagents as described herein. In certain embodiments, this may be achieved by filling a fluid column comprising a polymer sleeve and sealing the sleeve at intervals such that the divisions in the sleeve comprise the type and volume of a particular reagent in the apparatus and form sachets (including, for clarity, pouches). In certain embodiments, the sachets may contain different reagents, or may be formed or attached in such a fashion that they comprise a separate component. In certain embodiments, the sachets are stable at room temperature for at least about 7, 14, 21, 30, 45, 60, 90, 180, 365 days or more. In particular embodiments, these divisions may be heat sealed, induction sealed, sealed with adhesives, ultrasonically welded, a mechanical seal may be achieved with compression that comprises a part of the apparatus, or other methods that provide a sealed compartment suitable to the application as will be apparent to the practitioner.

In certain embodiments where both liquid and dry components are contained in the same cartridge, or closely positioned to a cartridge, the porosity of and gaseous transit properties of the material are important to consider. In some embodiments, the materials may be treated with a coating or finish to reduce gas transfer, for example, waxes, adhesives such as urethane, thermoplastics, foil encapsulation and other techniques and materials that will be apparent to the practitioner. In other embodiments, the treatment is a degassing, heating or vacuum step (or other methods sufficient) to dry the material, for example, by heating to about 75% of the working temperature of the material (or other embodiments, about 10%, 25%, 50%, 90%, 100% or above) and holding for a period of at least about 10 seconds, 30 seconds, 1 minute, 5 minutes, or more. In some embodiments, a vacuum (including a partial vacuum) may be used alone or in conjunction with such treatment, or a non-reactive gas of low relative humidity, for example, below about 20%, 10%, 5%, 1% or less, may be used to facilitate drying. In particular embodiments, the material may be resistant to gaseous transfer, for example a metal, such as a foil, or the vessel may be formed of the material. In other embodiments, the material may be shaped such that the travel path through which a gas would need to transit is extended. In particular elements, the travel bath is at least about 2 mm, 3 mm, 5 mm, 7 mm, 10 mm or more.

In particular embodiments, these sachets may be adhered to a portion of the apparatus that is in communication with one or more fluid paths. In certain aspects, the one or more fluid paths may become in communication with the interior of the sachets during the operation of the apparatus. In some embodiments, the adherence of the sachets to other portions of the apparatus may be facilitated by an adhesive. In other embodiments, the sachets may include an integrated port to allow adherence. In some embodiments, the port may include a valve, for example a check valve allowing fluid flow in only one direction (for example an umbrella, ball, or duckbill valve, among others), or a valve that allows fluid flow in more than one direction, for example a bidirectional valve, or a valve that allows fluid to flow in multiple directions. In particular embodiments, the sachet does not include a valve or port and may be pierced or violated to establish a fluid flow.

In some embodiments, the adherence of the sachets may be effected by tape, for example VHB tape. In certain embodiments, the tape or adhesive may form a ring or donut which may comprise a putative or actual fluid channel. In other embodiments, the sachets may not directly communicate with other fluidic channels, but may be contained within a structure that does communicate, for example a pocket, well, or vessel.

In certain embodiments, the cartridge elements may be sealed by use of heat, induction, ultrasonic welding, or other techniques familiar to the practitioner or described herein. In specific embodiments, a sealing plate is used to contact a sealing material to a substrate to ensure firm contact prior to sealing. In particular embodiments the sealing plate is used in conjunction with heat or induction to seal the cartridge elements. In other embodiments, the sealing plate is transparent and is used in conjunction with a laser. In particular embodiments, the sealing plate is a glass, for instance a borosilicate glass, or a fused silica, glass ceramic, heavy metal fluoride glass, meta-phosphate glass, or other glasses that will be apparent to the practitioner. In particular embodiments, the sealing plate is a polymer, metal, or other material suitable for the attachment and described herein. In certain embodiments, laser light passes through at least some of the sealing plate, which is providing at least some compression to bring the sealing material and substrate into proximity.

In some embodiments, the sealing may effected by use of a laser pulse to melt or fuse the materials or added materials to close an orifice and optionally trim the edges of the cartridge or related sealing surfaces. In other embodiments, the trimming is performed by a knife edge, separately cut, for example by laser, or pre-perforated or cut sheets may be used. In some embodiments, the laser is a CO₂ laser. In other embodiments, the laser is a YAG laser. In particular embodiments, an aperture may be sealed by compressing two opposed elements, optionally with heat, vibration, current, induction, ultrasound, or other sealing technologies that will be apparent to the observer. In certain embodiments, the laser power may be modulated such that it can both seal a sealing material (for example, a film, small pellet, disc or other shape that will be apparent to the practitioner) to a substrate (for example, a cartridge, valve body, port, or other aspect of the apparatus). In specific embodiments, the laser (understood herein to include other light or heat sources) may both seal the material to the substrate and additionally cut the sealing material to singulate the sealed features. In particular embodiments, the sealing material is polyethylene terephthalate (PET). In some embodiments, the substrate is polypropylene homopolymer or copolymer.

In select embodiments, the laser is a 100 W CO₂ laser using power settings of minimum and maximum power between about 3%, 4%, 5%, 6%, 7%, 8%, 9%, 10%, 11%, 12% or more and a speed of about 25 mm/s, 30 mm/s, 35 mm/s, 40 mm/s, 45 mm/s, 50 mm/s, 55 mm/s, 60 mm/s, 65 mm/s, 70 mm/s, 75 mm/s, or more, for example using a continuous wave setting. In certain embodiments, the laser head height may be 15 mm, 16 mm, 17 mm, 18 mm, 19 mm, 20 mm, 21 mm, 22 mm, 23 mm, 24 mm, 25 mm, or more (see Brown, N., et al., 2012.Non-contact laser sealing of thin polyester food packaging films. Optics and Lasers in Engineering, 50(10), pp. 1466-1473, or Ian Jones and Nicki Taylor Medical Plastics '98-12th International Conference, 7-10 September, Gothenburg and Addendum, July 2001., both incorporated herein by reference).

In some embodiments, the reagent cartridge incorporates a ROM, RFID, NFC, or similar technology to allow automated identification of the cartridge type by other components within the apparatus and optionally contain the parameters necessary and particular to the cartridge type to allow the remainder of the apparatus to function. This allows the interoperability of cartridges with the other elements of the apparatus without the need to update firmware, software, or settings on the apparatus when different cartridges are used.

In some embodiments, the reagents may be stored in the cartridge in gases other than natural ambient air. In certain embodiments, the relative humidity of the gas will be less than about 20%, 15%, 10%, 5%, or 1%. In particular embodiments, the gas is air, nitrogen, argon, a noble gas, sulfur hexafluoride, or other gases familiar to a practitioner. In certain embodiments, the gas is a at a pressure other than atmospheric pressure. In particular embodiments, the gas is below atmospheric pressure, for example a partial vacuum. In certain embodiments, the vacuum is at least about 50%, 60%, 70%, 80%, 90%, 95%, 99% or higher.

Nucleic Acid Extraction

In some embodiments, the apparatus contains a sample preparation module 200, an amplification module 300, and a detection module 400 disposed within an apparatus housing 100, capable in particular embodiments of accepting a raw biological sample and performing automated sample-to-result PCR or RT-PCR. In certain embodiments, the apparatus comprises only a single button, for example a momentary button 104 as shown in FIGS. 1-2, for user operation. In other embodiments, the apparatus comprises multiple user-actionable buttons. In particular embodiments, the apparatus does not include any user-actionable buttons, such as momentary buttons, toggle buttons, switches and the like. In specific embodiments, the apparatus comprises a zero-power state latch (as described elsewhere herein), which uses an actuator other than a momentary button, for example a mechanical force sensor, capacitance sensor, resistive sensor, orientation sensor, contact sensor, or other sensors that are capable of activation through a P-FET or in other embodiments, other electronic means, for example semiconductor or relay actuation, for example by N-FET, BJT (NPN or PNP), IGBT, or others that will be apparent to the practitioner. In certain embodiments, the actuator is a sample detection sensor, for example a resistive sensor. In this mode of operation, the embodiment may have zero or more user-operable buttons or switches, and may transition to operation from a zero-power state using a P-FET microprocessor latch, such that a user provides a biological sample (for example saliva) into the device, the zero-power state latch is activated by a current through the biological sample, the apparatus automatically detects whether there is a sufficient biological sample, performs sample-to-result PCR as described elsewhere herein, without further action required from the user.

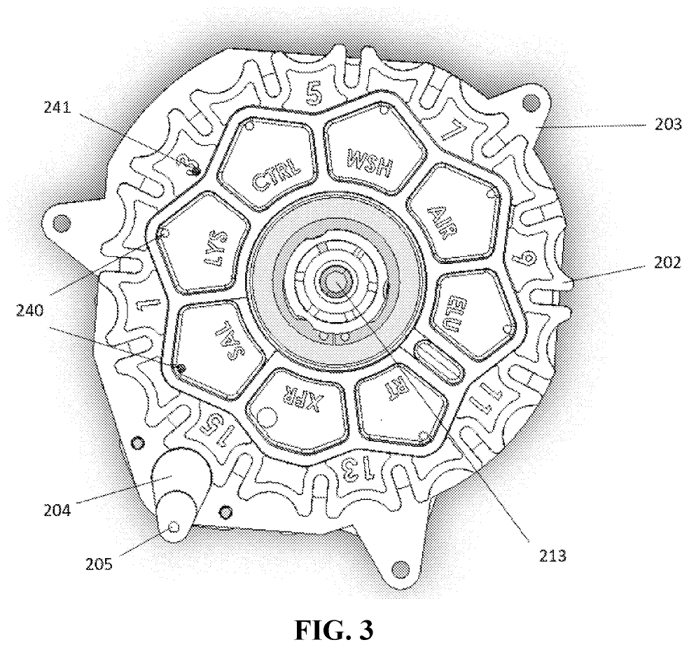

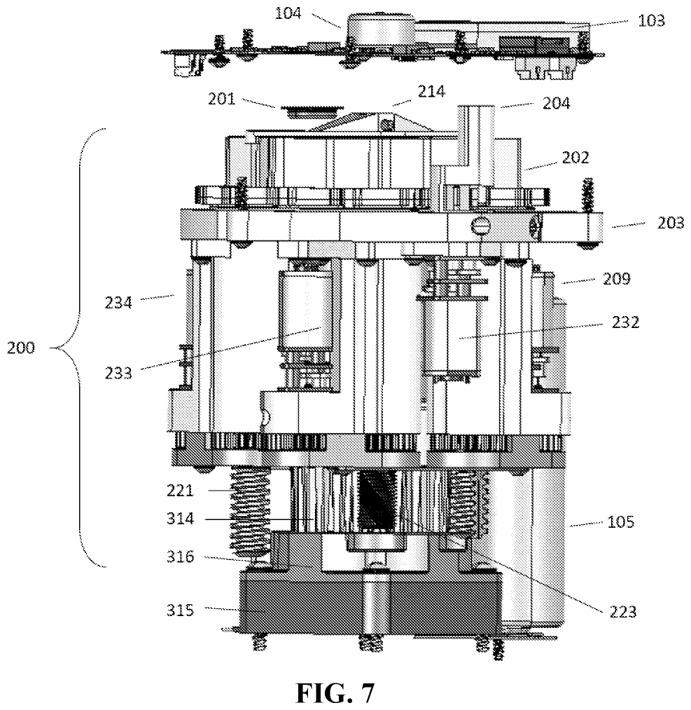

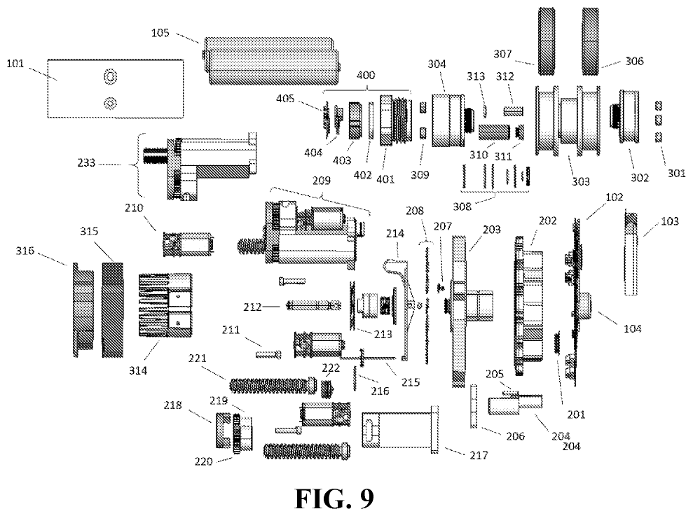

In certain embodiments, the sample preparation module 200 (see e.g. FIGS. 1 and 7) comprises a liquid (or predominantly liquid) sample collection funnel (which may optionally be integrated with the apparatus housing 100), which directs the sample to an optional prefilter 201 (see e.g. FIGS. 7 and 9), which in some embodiments may help to exclude certain fractions of the liquid sample as described elsewhere herein. In particular embodiments, the sample passes through the prefilter 201 and into a reagent cartridge 202. The reagent cartridge 202 may contain one or more wells of different sizes as show in FIG. 3. In certain embodiments, the liquid sample (for example, saliva) enters the well labelled “SAL” in FIG. 3 where it interacts with a sensor as described elsewhere herein. In some embodiments, the reagent cartridge 202 is located adjacent to a valve body 203 which may contain one or more apertures (for example piercer chamber apertures 240 or piercer lever apertures 241) (see e.g. FIGS. 3, 5 and 6).

In some embodiments, after the apparatus has determined that a sample of sufficient volume has been obtained (or in certain embodiments of the sensor, that it has the correct characteristics, for example, the correct resistance), the interface PCB 102 (see e.g. FIG. 9) may control a series of steps to extract nucleic acid material from the biological sample. In certain embodiments, first syringe assembly 233 will draw the biological sample into the first syringe, optionally pushing sample volume above the desired maximum sample volume back into the SAL well through the valve body the first syringe aperture, past the O-ring (or in some embodiments, a unitary O-ring) 208 (see e.g. FIG. 5), through the piercer chamber aperture 240 into well #0 (“SAL”) in the reagent cartridge 202. As shown in FIG. 7, the indexer 204, driven by indexer motor 232 will rotate the reagent cartridge 202 relative to the valve body 203 until the piercer assembly 234 is aligned under well #4 in the reagent cartridge 202. In certain embodiments, the indexer engages a gear, for instance a Geneva gear mechanism. The piercer 215 may be extended from the piercer assembly 234 through the valve body piercer aperture 250 (see e.g. FIG. 5) into the desired piercer chamber aperture 240 (in this instance, well #2 (“LYS”) which may contain a lysis reagent as described elsewhere herein.

In some embodiments, the piercer 215 (see e.g. FIG. 9) may puncture a seal covering the piercer chamber aperture 240 in the reagent cartridge 202, and continue to puncture a second seal covering the well (in this case, well #2 “LYS”) to produce a vent hole to allow the free movement of fluid into and out of the well of the reagent cartridge 202. In some embodiments, the tip of the piercer may be blunt, round, slanted, sharpened, or may incorporate multiple angles, including trocar tips such as pyramid tips, single blade, flat tip, dilating trocar tips, and other types of tips that will be familiar to the practitioner.

After the seals of the well have been punctured, the piercer 215 may be withdrawn by the piercer assembly 234, and the reagent cartridge 202 may again be rotated relative to the valve body 203 until the well which was punctured by the piercer is aligned with an aperture that corresponds to a syringe, in this case, the first syringe. The first syringe, which contains the desired volume of biological sample, may draw the lysis fluid in well #2 “LYS” into the first syringe by the action of first syringe assembly 233. Optionally, the contents of the syringe may be mixed by the action of first syringe assembly 233, for instance by moving the mixture into well #2 “LYS” and withdrawing it one or more times (for example about two, three, or more times). The mixing action serves to help homogenize the lysis mixture with the biological sample, increasing the speed of lysis and in some embodiments, obviating the need for a heating or incubation step.

In particular embodiments, the indexer rotates reagent cartridge 202 relative to valve body 203 until the piercer 215 is aligned with the next well in the reagent cartridge 202 to be processed, for example, well #4, “CTRL”, which may contain an exogenous internal positive control, as discussed elsewhere herein. In some embodiments, in a similar fashion to the operations performed on well #2 “LYS”, the piercer 215 may move to puncture and vent well #4, “CTRL” in the reagent cartridge 202, and the contents of the well may be mixed with the lysate from the previous step.

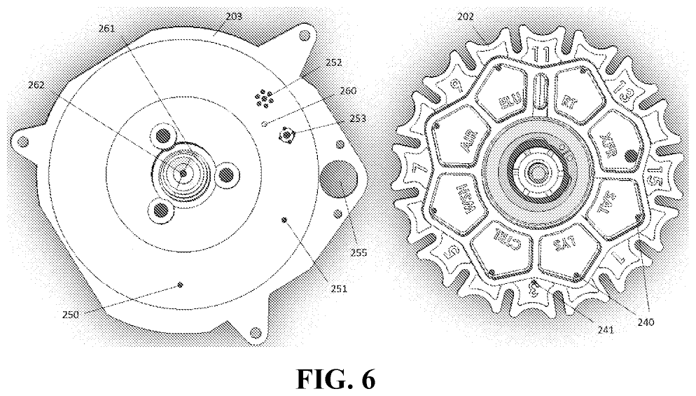

In certain embodiments, the homogenized or mixed lysate containing the biological sample, control, and lysis solution may be retrieved from a well, for example, well #2 “LYS” by the second syringe by the action of second syringe assembly 209. In some embodiments, the second syringe is collocated below the valve body check valve aperture 252, which may contain a check valve 207 as shown in FIG. 9 (for instance, an umbrella valve, duckbill valve, or other valves known to practitioners of the art). In some embodiments, the check valve 207 may allow fluid to enter into the second syringe, but prevent fluid from exiting the second syringe via the check valve 207. In particular embodiments, fluid in the second syringe may exit the syringe via a communicating channel to the valve body filter aperture 253. As fluid is pushed out of the second syringe, it passes through the filter 221 (see e.g. FIGS. 7 and 9) in the valve body filter aperture 253 (see e.g. FIG. 6), and interacts with the material within the filter, as described elsewhere herein. The fluid which passes through the filter 221 may be passed into a well in the reagent cartridge 202 with sufficient residual volume to be stored as waste fluid. It is preferable to select a well that is not needed in future steps to avoid contamination or mixing of incompatible reagents. For example, in some embodiments, the waste fluid may be stored in a previously used well, such as well #2 “LYS” or well #4 “CTRL” once the lysis or control fluids have been used.

In some embodiments, the lysate formed from the biological sample, control, and lysis solution may be passed through the filter 221 from the second syringe by the action of second syringe assembly 209, and the nucleic acids, including for example, DNA and RNA may adhere to the filter 221 by the action of chemistry of the lysis solution on the solid phase extraction media as described elsewhere herein. After the nucleic acids have adhered to the filter, in some embodiments, the indexer 204 may rotate the reagent cassette 202 relative to the valve body 203 and by action of the piercer 215, puncture well #6 “WSH” in the cassette 202 which may contain a wash solution as discussed elsewhere herein. The wash solution may be drawn into the second syringe by the action of the second syringe assembly 209 through the check valve 207, and after indexing the reagent cartridge 202 with the indexer 204, may be passed through the filter 221 to wash the chaotropic salts and other contaminants through the filter, for example into a previously used well as described herein. In certain embodiments, it is preferable to perform more than one wash, including dividing the total volume of wash to be used into one or more wash steps such that any residual chaotropic salt or contaminant present (in some embodiments) in the second syringe is diluted with each wash step and the final concentration of the chaotrope in the amplification reaction is minimized as described elsewhere herein. In particular embodiments, the action of the syringe plunger 222 (see e.g. FIG. 9) within second syringe assembly 209 assists in the liberation of residual chaotrope from with the body of the second syringe and other areas with which it is in fluid communication by, for example, mechanical agitation, turbulence, and dilution.

As described elsewhere herein, in particular embodiments, it may be advantageous to minimize the amount of wash present in the amplification reaction. In particular embodiments, this may be realized by passing air, including heated air in some embodiments, through the portions of the apparatus containing wash, for example the second syringe or filter 221. In specific embodiments, second syringe assembly 209 may comprise a heater. In certain embodiments, a heater may be located in proximity to the filter 221, for example in the valve body heater groove 254 shown in FIG. 5. In other embodiments, air may be passed through filter 221 to expel residual wash from the filter. In specific embodiments, an absorptive material, for example cotton, filter paper, or other materials that will be familiar to a practitioner in the art, may be present in well #8 “AIR” of the reagent cartridge 202, which may aid in the absorption of the wash. In certain embodiments, air may be passed through filter 221 one or more times.

In particular embodiments, the residual wash present in the second syringe or filter 221 may be further reduced by the use of a non-ionic, amplification-compatible wash (an “oil wash”), as described elsewhere herein. In particular embodiments, the reagents for the oil wash may be located in one of the wells of the reagent cartridge 202, for example, well #10 or well #12. In certain embodiments, the oil wash may be drawn into the second syringe after seal puncture by the piercer 215 similar to prior embodiment steps. The oil wash in the second syringe mechanically displaces residual wash. In particular, the lighter density and relative immiscibility of the oil wash with the previous washes, particularly alcohol based washes, tends to liberate the alcohol and wash components to float on top of the oil wash. In certain embodiments, the oil wash may pass through the filter 221, similarly displacing residual wash, including alcohol wash in the filter, decreasing the absolute quantity of contaminants and alcohol in the final amplification reaction, for example less than about 5%, 4%, 3%, 2%, 1%, 0.5%, or less. Similar to the division of washes described earlier which tend to displace or dilute chaotropes at a more than additive-rate based on the volume of the wash, the use of one or more oil washes tends to displace or dilute the wash, including alcohol washes, and the division of the oil wash into several fractions, in some embodiments has a more than additive decrease of the residual alcohol wash.

In some embodiments, after one or more washes and zero or more oil washes, the nucleic acids present in the filter 221 may be eluted into the amplification chamber. In certain embodiments, the indexer 204 positions the reagent cassette 202 relative to the valve body 203 such that the egress from the valve body filter aperture 253 (see e.g. FIG. 6) passes between the reagent cassette 202 and the valve body 203 within the bounds of the transfer O-ring 242 (see e.g. FIG. 5) and enters the valve body transfer aperture 260 (see e.g. FIG. 6). Within the valve body transfer aperture 260, the eluate flows through a channel within the valve body 203 and exits through an aperture into the valve body chamber holding 261, and then enters the amplification chamber of the amplification module 300 through valve body chamber ingress 262.

As shown in FIG. 9, in certain embodiments the amplification chamber comprises a hot end of the amplification chamber 302 which is mated to a thermal break 303. The hot end of the amplification chamber 302 may comprise one or more hot end bistability magnets 301. The thermal break 303 is also mated to the cold end of the amplification chamber 304, which similarly may comprise one or more cold end bistability magnets. In certain embodiments, the reaction mix may be disposed the chamber, for example, in lyophilized form. In some embodiments, the bistability magnets provide orientation independent displacer piston operation as discussed elsewhere herein. Disposed within the amplification chamber in some embodiments, is a magnetic displacer piston comprising a piston body 310 and piston cap 311 which contain one or more piston magnets 312. The piston body 310 and piston cap 311 may be sealed with a variety of approaches as described elsewhere herein, including ultrasonic welding. In particular embodiments, the bistability magnet may be a rare-earth magnet, for example, a N52, N50, N48, N45, N42, N40, N38, or N35 neodymium magnet. In some embodiments, the length, width and height of the magnet may be about 2 mm, 3 mm, 4 mm, 5 mm, 6 mm, 7 mm, 8 mm or more.

Also disposed within the amplification chamber in some embodiments may be lyophilized reagents 313 (for example, a bead or pellet), and surrounding the exterior of the amplification chamber in some embodiments may be one or more hot end heaters 306 and zero or more cold end heaters 307. The cold end of the amplification chamber 304 may also be located in proximity, and particularly thermal proximity, to a heat sinking element, for example in some embodiments, the cold end heat sink 314. Heat management of the apparatus may include active cooling, and in these embodiments, an active cooling component, for instance a fan 315 and fan holder 316 may be beneficial, particularly in conjunction with a cold end heat sink 314 when the heat sink includes features to promote air passage through or near the heat sinking features, for example, thermally conductive areas of high surface area in thermal proximity to the components.

In certain embodiments, the apparatus may comprise a detector, for example an optical detection module 400. In particular embodiments, the detection module 400 comprises an optical housing 401, a transparent window 402, and one or more filters comprising a filter assembly 404 disposed within an optical scaffold 403 which functions to limit cross-talk among the elements. In specific embodiments, the filter assembly 404 adjusts the passable wavelengths from light sources (for example, LEDs) on the optical PCB 405, as well as the wavelengths of light emitted from the amplification chamber as part of a fluorescence reaction through the transparent window 402 to be interpreted as the signal from an amplification reaction, for example a PCR or RT-PCR reaction.

In particular embodiments, a user of the apparatus may provide a biological sample through the sample preparation model (for example through the aperture in the apparatus housing 100), which passes through the prefilter 201, is controlled by user actions of the momentary button 104, is processed to extract nucleic acids by the sample preparation module 200, amplified in a reaction (for example a PCR or RT-PCR reaction) by the amplification module 300, is detected by the detection module 400, and is interpreted by the firmware present on one or more PCBs (for example, the interface PCB 102 or the amplification PCB 101) and provides results (whether in a blinded fashion or not) via the display screen 103, which may be an LCD screen in some embodiments.

Amplification

In some embodiments, the apparatus comprises an amplification chamber. In certain embodiments, the amplification chamber is capable of containing fluid in a range of temperatures from about 0° C. to 100° C. or more. The chamber may be constructed of one or more materials and may include one or more sensors. In some embodiments, the chamber material is a metal, a polymer, or a glassified material. In some embodiments, the metal is a non-ferrous metal or alloy, such as aluminum, noble or precious metals such as gold, silver, or platinum, or other metals or metal alloys, for example tungsten or titanium and related alloys. In certain embodiments, non-metallic thermally conductive material such as a pyrolytic graphite sheet (for example used in conjunction with a matrix such as silicone or other resins such as “Graphite-PAD”) as a thermal interface material.

In certain embodiments, the polymer is polypropylene, polyethylene, acetal rod or resin, polystyrene, acrylic, PDFE, PMP, PVC, polycarbonate, PFA, PCTFE, PSU, or other polymers that will be apparent to the practitioner. In some embodiments, the aluminum is a wrought or extruded metal. In other embodiments, the aluminum is a cast metal. In certain embodiments, the aluminum is a 1000 series alloy. In other embodiments, the alloy is of a different series, for example a 2000, 3000, 4000, 5000, 6000, 7000, or 8000 series alloy. In some embodiments, it is preferred to avoid alloys containing copper, iron, chromium, or zinc, or to select alloys with low percentage contents of these metals (for example, below about 0.5%, 0.3%, 0.1% or lower percent by weight). In particular embodiments, the glassified material is silicate glass, for example a borosilicate glass or fused quartz. In other embodiments the glass is a soda-lime, aluminosilicate, ceramic glass, or other glasses that would be apparent to a practitioner of the art.

In certain embodiments, the chamber is coated or treated to improve the performance of the amplification reaction or detection characteristics. In certain embodiments, where a metal chamber is used, the chamber may be treated by anodization, passivation, or the deposition (plating) of a secondary metal. In specific embodiments, the chamber may also be coated or lined by a metal, glass, polymer or chemical coating, for example, PTFE (including glass-filled PTFE), stainless steel (for example SS 304), carbon fiber or other forms of carbon, borosilicate glass, silicone rubber, enamels (for example, Cerakote), various glues, such as urethane-based glues (including Nano 470), adhesives (for example, Henkel™ PC4400), PDMS, conformal coating (for example, Parylene, including Parylene-C), PDVF, polycarbonate, and other compounds that will be apparent to the practitioner. In some aspects, an epoxy-based coating or glue may be used (for example, Torr-Sea), or an ester-based coating or glue may be used (for example, cyanoacrylates), however care should be taken to achieve complete curing to avoid adversely affecting subsequent amplification reaction chemistry.

The coating or liner may be applied via injection molding, extrusion, as a separate insert, via vapor deposition, in liquid form, as a dry powder, as a heat-activated process, as a UV-cured process, as a drying/evaporative process, or anodization or passivation process. In some embodiments, the, use of anodization may create microstructures which tend to absorb reaction components in amplification reaction chemistry. Additionally, in some embodiments, these microstructures and porosity characteristics may contribute to autohydrolysis of hydrolysis probes used in the detection chemistries of some amplification reactions (for example, TaqMan). An alternative embodiment is to use a different passivation process, such as ozone treatment or plasma electrolytic oxidation, which creates a less porous microstructure and in certain embodiments has improved performance.

In particular embodiments, a combination aluminum and polymer chamber is used that does not have a coating or liner specifically applied. In specific embodiments, the aluminum alloy is 6061 and the polymer is Delrin or other acetal resins. In some embodiments, the aluminum chamber portions are divided by the polymer portion in such a way that the polymer provides a thermal break or insulative function to mitigate the flow of heat from one of the aluminum portions to the other. In certain embodiments, a larger polymer gap will include thermal isolation of the aluminum chamber portions, but the larger gap will contribute to a larger surface area of the polymer within the amplification chamber. In some aspects, this may decrease the surface area available for metal-based conductive heating of the fluid in the amplification chamber. In other aspects, gas (including air and water vapor) transit of the polymer is increased with an increased surface area, which may adversely affect the amplification reagents, for example if they are stored in lyophilized form within the amplification chamber. In some embodiments, it is therefore advantageous to determine the proper polymer gap, which is also subject to other constraints of engineering and manufacture that will be apparent to the practitioner. In specific embodiments, the polymer gap between the aluminum portions is about 1 mm, 2 mm, 3 mm, 4 mm, 5 mm, or more. In particular embodiments, the apparatus comprises a device which has a maximum tolerance of at least 0.002″, 0.003″, 0.005″, 0.006″, 0.007″, 0.008″, 0.009″, 0.01″ or more.

Displacer Piston

In certain embodiments, the amplification chamber comprises a displacer piston. The displacer piston moves between two or more positions to cycle a fluid between two or more temperatures. In certain embodiments, the displacer piston is magnetic, or contains magnetic elements. In other embodiments, the displacer piston is driven by a mechanical linkage. In particular embodiments, the apparatus may comprise additional magnetic elements which impart “bistability” to the displacer piston's position such that in the absence of additional forces, the piston will tend to locate itself to one end of the amplification chamber or the other or will tend to remain at one end of the amplification chamber or the other until another force compels the displacer piston to move from that position. In certain embodiments, this bistability operates irrespective of orientation, or irrespective of a subsegment of orientation, for example in an arc of 180° from horizontal to vertical to horizontal along the axis of piston or amplification chamber. In particular embodiments, an electromagnetic field in the apparatus is sufficient to overcome the bistability to toggle the piston position between ends of the amplification chamber.

In some embodiments, the electromagnetic field is generated by an actuator, for example, one or more wire coils which impart a force on the magnetic elements such as the displacer piston. In some embodiments, it may be advantageous to colocate the coils over the heating portions of the amplification chamber to allow recapture of waste heat from coil activation. In certain embodiments, the coils may be controlled by a microprocessor, including by intermediary components such as an H-bridge or motor driver. In some embodiments, the displacer piston performs a mixing function, for example to help with the resuspension of a lyophilized or dried pellet, or to homogenize the reaction components, for example the amplification reaction chemistry, including related chemistries such as reverse transcriptases or fluorescence detection chemistries. In particular embodiments, the mixing function increases the efficiency of the reaction at least about 10%, 25%, 50%, 75%, 100%, or more. In certain embodiments, the movement of the displacer piston may be performed in a vibratory mode at high oscillation (for example, greater than 1 Hz, 2 Hz, 3 Hz, 5 Hz, 10 Hz, 25 Hz, 50 Hz or more) to cause more active mixing or cavitation. This mode of piston action is useful in some embodiments to disrupt gas bubbles or dislodge the piston should it become stuck in one position (for example by adherence to a partially dissolved lyophilized pellet).

In particular embodiments, the increased efficiency of the reaction allows rapid heat exchange into the amplification reaction fluid, for example for a PCR reaction. Although high ramp rates for PCR have been reported, typically 3-5° C./s or higher, or in some case 8-10° C. or more, in practice these rates are maximum ramp rates, and average sample ramp rates are lower. In addition, traditional PCR reactions are typically quite small volumes, for example about 20 μL (and ranging from about 1-50 μL) in most applications. Thus, the thermal ramp rates reported for traditional PCR apparatuses are predicated on a small volume, in other words, for example, 3° C./s for a volume of 20 μL. In some embodiments of the apparatus disclosed herein, the volume of the reaction is about 500 μL. As such, the ramp rate is best calculated as a function of volume. For example, in the aforementioned example, the “volumetric ramp rate” may be described as 3*20=60° C.·μL/s. Very high ramp rate PCR machines typically achieve maximum average sample volumetric ramp rates of 250° C.·μL/s. In some embodiments of the present disclosure, the volumetric ramp rates are greater than about 300° C.·μL/s, 400° C.·μL/s, 500° C.·μL/s, 600° C.·μL/s, 700° C. μL/s, 800° C.·μL/s, 900° C. μL/s, 1000° C.·μL/s, 1100° C. 1200° C.·μL/s, 1300° C.·μL/s, 1400° C.·μL/s, 1500° C.·μL/s, 1600° C.·μL/s, 1700° C.·μL/s, 1800° C.·μL/s, 1900° C.·μL/s, 2000° C.·μL/s, or higher.

In specific embodiments, the displacer piston is a polymer that contains a magnet. In certain embodiments, the piston is ultrasonically welded. In other embodiments, it is injection-molded, extruded, glued or machined. In particular embodiments, the surface of the piston is treated to provide a uniform surface with a roughness that varies by no more than about 100%, 50%, 10%, 5%, 1%, or less or is less than about 5 μm, 4 μm, 3 μm, 2 μm, 0.5 μm, 0.25 μm, μm, 0.05 μm, 0.025 μm, or less.

Chamber Thermal Control

In some embodiments, the amplification chamber includes one or more heaters. In certain embodiments, the heater are resistive heaters or Peltier heaters. In other embodiments, the heat source may be electromagnetic induction, adiabatic, microwave, radioisotope, sonic, friction, chemical, geothermal, user/body heat, or solar methods of heating. In particular embodiments, a control loop, for example a control loop using one or more elements of proportionality, integration, and derivation (PID) to maintain thermal control as will be apparent to a practitioner in the art. In certain embodiments, only proportionality is used as a control metric and heat buffer in the amplification chamber allows for adequate thermal control without the additional computation time of the other elements of PID-like control loop structure. In some embodiments which use a displacer piston or other space-domain approaches for thermocycling amplification reactions like PCR, it may be advantageous to preheat or precool one side of the amplification chamber while in the opposite half-cycle of amplification. This preconditioning improves ramp rates and may be performed at a magnitude of about 1° C., 2° C., 3° C., 4° C., 5° C. or more, constrained by the risk of flash boiling (in the case of preheating), or non-specific amplification (in the case of precooling), as well as thermal steal either directly between ends of the amplification chamber or through the fluid if the temperature gradient exceeds the rate of the heating and cooling elements of the amplification chamber. In certain embodiments, for example when active cooling is used, the ratio of the masses of the hot and cold ends of the amplification chamber may be about 1:1, 0.5:1, or 2:1. In particular embodiments where passive cooling is used, the ratio of mass between the hot and cold end may be about 0.1:1, 0.25:1, 0.5:1, 0.75:1, or 1:1. In some embodiments, the surface are of the cold end (optionally including a heat sink) may be about 1.1:1, 1.25:1, 1.5:1, 1.75:1, 2:1, 2.5:1, 3:1, or more.

In particular embodiments, the heater is a resistive heater in contact (optionally in conjunction of a thermally conductive compound) with the amplification chamber. In certain embodiments, the thermally conductive compound is an adhesive. In specific embodiments, the one or more heaters include temperature sensing elements, for example thermistors or thermocouples. In specific embodiments, the amplification chamber will include one or more thermal sensors, for example thermistors or thermocouples. In particular embodiments, the resistive heater is a strip heater including thermal sensors. In some embodiments, the thermal sensors are within about 5 mm, 4 mm, 3 mm, 2 mm, 1 mm, 0.5 mm, 0.4 mm, 0.3 mm, 0.2 mm, mm or less from the interior of the amplification chamber.

In certain embodiments, the heaters are controlled by one or more microprocessor. In some embodiments, an H-bridge is used to control the heater. In particular embodiments, a transistor or similar electrical control element is used to control the heater. In certain embodiments, it is advantageous to establish a sequence control to prevent current “shoot-through” whereby the current may pass through the transistors but bypass the heat load. In other embodiments, a state machine in the one or more microprocessors allows direct control of the heaters (without the use of motor-driver control chips, which would otherwise handle the timing and manage disallowed states).

In some embodiments, the thermistors have an accuracy of about 0.1%, 0.5%, 1%, or 2%. In other embodiments, the thermistors are not calibrated prior to their use in the apparatus. In particular embodiments, a lookup table or formula may be used to compensate for thermally-dependent performance of the thermistors, for example, linear interpolation between entries of a lookup table may be used. In other embodiments, a search approach, for example a binary or log(n) search may be used to iterate during a lookup function.

In certain embodiments, the amplification chamber comprises two ends, which may be at different temperatures. In particular embodiments when there is one end at a lower average temperature than the other, and where the maintenance of the average temperature of one or more ends is desired, it may be advantageous to include active cooling of the side with lower temperature. In specific embodiments, the active cooling may be a fan, liquid cooling loop, heat pipe, Peltier, or other thermal regulatory device or devices that will be apparent to the practitioner including chemical, liquid or phase change, adiabatic, and the like. In other embodiments, cooling is passive, and may be implemented using specific geometries, environmental exposure control, heat sink exposure, nanotubes, and other structures apparent to the practitioner. Both active and passive cooling embodiments may be used in conjunction with one another and with structural elements designed to improve thermal transfer, for example heat sinks and heat straps may be used in conjunction with fans to obtain synergistic effects that are more than additive.

Detection

In certain embodiments, the apparatus comprises a detector. In some embodiments, the detector is an optical detector capable of detecting fluorescent light emitted from the reaction. In certain embodiments, the apparatus comprises one or more LEDs. The wavelengths of the emission maximum of these LEDs may be correlated with various fluorescent dyes, including, for example, hydrolysis probes or molecular beacons. In certain embodiments, it is advantageous to drive the LEDs at currents which exceed their rated capacity. In some embodiments, the LEDs may be driven at more than about 1X, 2X, 3X, 5X, 10X, 15X, 20X, 100X, 250X, 500X, 1000X or more of their rated capacity. For example, a typical LED may have a maximum rated current of 350 mA for 100 ms, but may be run at 10 A for 1 ms. To avoid destruction, damage, changes in optical performance, variability/noise, or optical attenuation (bleaching) of the LED, in particular embodiments, the duty cycle may be adjusted such that a brief pulse of current exceeding the rated capacity of the LED is used (a “superpulse”) which permits the LED to dissipate heat at a rate such that for any particular duty cycle the temperature of the LED does not exceed about 50° C., 75° C., 100° C., 125° C., 150° C., 200° C. or more, or in some embodiments, any temperature that could cause changes in the operation of the LED that would adversely affect the system. In some aspects, a heat sink may be used to assist in the regulation of the temperature of the LED and mitigate the risk of exceeding the maximum non-destructive operating temperature of the LED. In some embodiments, the duty cycle of the LED is as much as 100%, or less than about 5%, 4%, 3%, 2%, 1%, or less. In certain embodiments, the pulse length is less than about 25 ms, 15 ms, 10 ms, 5 ms, 1 ms or less.

In some embodiments, the apparatus is tuned to improve the performance of an LED during a superpulse. In particular aspects, LEDs, when experiencing current have a variable response rate, such that an intensity ramp develops over the initial moments, followed by a relative intensity plateau, and then finally an intensity attenuation driven predominantly by thermal effects. To tune the performance of a superpulsed LED, in specific embodiments the apparatus uses an algorithm to measure the relative intensity of the LED at intervals from current initiation until the onset of decay. In some aspects, the time delay is about 3 ms, 5 ms, ms, 15 ms, 20 ms or more. In particular embodiments, the measurement is performed in concert with a detector. In certain embodiments, the detector is contained within the apparatus. LED performance may be affected by non-linear current dynamics, heat transfer and sinking, and temperature. In specific embodiments, the variation between LED profiles of different sources or types, or variation among individual components may be normalized by the use of an adaptive firmware routine. In particular embodiments, the adaptive firmware routine may be used to autotune each device automatically.

In specific embodiments, the adaptive firmware routine is to activate the LED with a superpulse, measure the light intensity with a detector, for example an analog to digital optical detector, and sample the light at intervals, for example once per millisecond, and to continue sampling until two or more successive points have increased in intensity and following this, that two or more sample points have decreased in intensity. Alternatively, a static number of points (or static at particular temperature values in some embodiments), for example about 30 samples, or in other embodiments, about 10, 25, 50, 75, or 100 samples may be used to generate the intensity data for the adaptive firmware routine. The index of the interval of highest intensity between these two events (in the case of a static point selection approach, the first and last points of the static range each constituting an event), or alternatively, an average, including a weighted average of two or more points between these two events is selected as the tuned sampling point index for future measurements using the LED. In some embodiments, the information and measurements from the sampling may be used to calculate a pulse width for the optical system. In particular embodiments, the pulse width approximates the width of the plateau. In other embodiments it is a subset of the indices of the plateau phase, for example a subset constituting 10%, 25%, 50%, 75%, 90% or more of the plateau. In systems with multiple LEDs, this may be repeated for each LED to be tuned, and the process may be verified or reverified when the optical system components have reached a higher, or preferably, steady-state temperature. In certain embodiments, the current or temperature of the LED may affect the emission frequency or frequencies.

In some embodiments, the use of an LED with a linear or near linear relationship between temperature and wavelength may be used in conjunction with a predictive routine to compensate for the wavelength shift, for example, using a table lookup or formula to establish a translational map between LED current and wavelength shift. In some embodiments, the shift in wavelength is less than about 25%, 15%, 10%, 7.5%, 5%, 3%, 2%, 1%, or less. The translational map may include variables or table columns for performance by temperature. In other embodiments, for example where optical filters are employed or the adaptive firmware routine is used, the maximal response index during the sampling interval may be selected based not only on the maximal response for the particular LED, but in the case of systems with multiple LEDs, also based on the (preferably minimal) responses of other LEDs to mitigate or prevent optical cross-talk. In some embodiments this may rely on selecting the high and low values, average of several points, or weighted averages, again, in particular embodiments, correlated by temperature. In particular embodiments, this adaptive firmware routine may be run during the manufacturing process, for example a built-in self-test (BIST). In other embodiments, it may be performed during device use prior to detecting optical signal data.

As LED performance varies by temperature, in certain embodiments, the apparatus or certain components within the apparatus, may be preheated before data are collected, including calibration or autotuning data. In other embodiments, elements such as temperature controlled oscillators, thermistors, thermocouples, or other temperature measurement methods may be used in the apparatus to compensate for thermal changes in the apparatus that might otherwise contribute to variability or negative performance. In specific embodiments, the preheating function may mimic the intended LED usage duty cycle. In other embodiments, the apparatus may include temperature sensors or heat sinks or heat straps such that the temperature-sensitive elements of the apparatus (for example, resistors in optical circuits) may be heated to a known or approximated temperature corresponding to the desired stable thermal environment representative of the environment during optical detector use. In certain embodiments, the apparatus may use data from temperature sensors directed to the ambient environment to correct for operating conditions that are aberrant from the apparatus temperature conditions. In particular embodiments, the deviation of such aberrant temperature states may be incorporated to a controller, for example a controller incorporating elements of a PID controller or similar or alternative feedback routines that will be apparent to the practitioner. In certain embodiments incorporating a variable gain element, the preheating parameters may be modified to reflect the amount of gain the detector is tuned to (as discussed elsewhere herein), where in some embodiments, increasing gain may require additional downward temperature compensation. In specific embodiments, the degree of compensation may be calculated using a table or formula comprised by the apparatus.

Auto-Tuning

Traditional apparatuses for nucleic acid amplification (including PCR) that incorporate optical detection elements are typically complex and expensive. This has been a significant barrier to the development of low-cost, highly-sensitive optical detection systems for nucleic acid amplification. The present disclosure addresses these limitations and describes optical detection systems, methods, and apparatuses for PCR that do not require individual radiometric calibration of hardware elements. In some embodiments, the apparatus comprises an auto-tuning routine that automatically calibrates the appropriate LED intensity and gain settings for the apparatus. In certain embodiments, the routine may be preceded by an adaptive firmware routine to select optimal LED timing as described elsewhere herein. In some embodiments, the auto-tuning may be effected by taking initial measurements at the beginning of an amplification reaction. For thermocycling amplification reactions, these initial measurements may be taken at cycle 1, 2, 3, 4, or 5, or principally any cycle where the amount of fluorescence can be considered background fluorescence. In particular embodiments, initial measurements may be done prior to PCR amplification cycling, for example during the reverse transcriptase or temperature ramping phases. In specific embodiments, the reverse transcriptase or cold-side PCR temperature may be selected to allow like temperatures in adjacent phases to permit earlier initial measurement collection. Depending on the reaction, this range may extend to about cycle 10, 12, 15 or more. A nested loop approach may be used to scan across the range of gain and LED conditions. In some embodiments, the resolution of these two variables may be greater than about 2, 6, 8, 12, 16, 32, 64, or more samples per range element. In particular embodiments, the gain may be controlled by a variable potentiometer (or rheostat), including one that is software controlled, through for example, and I2C bus. In some embodiments, the communication speed controlling the gain and LED intensity may be different. In these instances, it may be advantageous to nest the search loops such that the faster communication protocol is paired with the variable (LED or gain) with a greater resolution.

In some embodiments, the data collected by the initial (for example, nested loop) scan of gain and LED intensity may be evaluated against the response from the detector. In certain embodiments, the initial scan parameters may be adjusted during the scan by using the response from the detector to adjust detector gain and/or LED intensity based on the measured or calculated analog to digital values (ADC ticks) such that the measured or calculated detector response falls within a desired band in the range of the detector. In particular embodiments, the range may be a central percentage, for example the range from about 35% to 60% of the full detector range. In specific embodiments, for example with an ADC range of 0 to 4096, a range of about 1500-2500 may be used. In other embodiments, the range may be adjusted to a smaller or larger range, and may be located towards the center, or top or bottom of the detector. In certain embodiments, the positioning of the range may be determined by the fluorescence delta (between baseline and fully digested probe) of the fluorescence dyes. In other embodiments, the signal to noise ratio (SNR) of the optical system may be used to adjust the range, such that high noise systems may require a large number of samples, or the range may be moved toward the high or low end of the detector limits (depending on the system bias and reference voltage, as will be apparent to the practitioner).

In particular embodiments, when a putative gain and LED intensity combination yields an ADC value outside the desired band, the variable element (for example of a particular loop) is adjusted to decrease or increase the measured or calculated ADC value until it is within the desired band. In some embodiments, each putative gain and LED intensity combination that falls within the desired band (or in certain embodiments, a subset of them) is recorded in a solutions table. The number of combinations to evaluate is affected by the self-heating thermal aspects of the optical detector. In practice, a solution set of fewer than about 32 solutions is adequate for some embodiments, particular in embodiments which have undergone an adaptive firmware routine to tune LED timing as described elsewhere herein. In other embodiments with greater resilience to self-heating, a greater number of solutions may be retained, however the utility of these solutions is limited by the number of solutions that may be used during detection, which is depending on the duty-cycle for the particular embodiment of the apparatus. Once the solution set has been established, it is used as the basis for detection in the apparatus. In some embodiments, this use may be modulated or altered by additional data available to the apparatus, for example the temperature of various temperature-sensitive components. In certain embodiments, the optical scan time during detection using the solution set less than about 100 ms, 50 ms, 25 ms, 10 ms, 5 ms, 3 ms, 1 ms, or less. In particular embodiments, the short scan time obviates the need for heat strapping or sinking of the optical detection elements.

Efficiency

The radiant flux generated by the fluorescence of nucleic acid amplification chemistry is low in absolute terms, and as a result, to address the constraint of a low number of photons reaching the detector, traditional optical detection systems for nucleic acid amplification typically use lenses, dichroic mirrors, and precisely calibrated optical hardware to allow maximize SNR and allow reliable detection. The present disclosure describes methods, apparatuses, and systems that allow for reliable, low-cost detection without the approaches typically required of traditional optical detection systems for nucleic acid amplification. In some embodiments, a baseline fluorescence is compared to fluorescence during the course of an amplification reaction in real-time. In particular embodiments, smoothing algorithms, for example regression analysis or outlier identification may be used to pre-process data. In certain embodiments, a minimum threshold or minimum positive slope may be used when identifying deviations from the baseline.

In some embodiments, low absolute optical signal may be increased in electrical circuits by increasing gain, for example through the use of an operative amplifier. However, the resistance required to increase gain to the level necessary to directly detect the fluorescent signal is quite large. In some embodiments, the gain is greater than about 100k, 150k, 250k, 500k, 1M, 2M, 3M, 4M, 5M, 6M, 7M, 8M, 9M, 10M, 25M, 50M, 100M or more. The resistance needed to support such a gain is therefore also quite large. Very large gains (and resistances) bring into play parasitic resistances that exist on circuit boards. These parasitic resistances may exist due to impurities and variations in the hardware or manufacturing processes, for example contaminants, humidity, temperature, or other factors which affect resistance that will be apparent to the practitioner. This results in current flowing through unintentional paths and produces an unstable or inconsistent noise and harmonics induced by the oscillating (functional) system gain which might be erroneously interpreted as signal in some cases. The particular threshold of gain and resistance that this might occur with depends on the particular elements on the circuit board, however in some embodiments, any gain above a certain threshold (for example 1M) may experience such noise.

To achieve stability and consistency, some embodiments comprise a constant-current T-network transimpedance amplifier (converting current to voltage) by reconfiguring the requisite resistance into a Thevenin equivalent (see J. E. Brittain, “Thevenin's theorem,” in IEEE Spectrum, vol. 27, no. 3, pp. 42-, March 1990, and Karki, Jim, “Fully Differential Amplifiers Applications: Line Termination, Driving High-Speed ADCs, and Differential Transmission Lines,” in Texas Instruments Application Report SLYT143, February, 2001 incorporated herein by reference). In certain embodiments, the current source may be driven directly by a microprocessor. In other embodiments, it may be driven by an intermediary, for example a driver circuit or relay bank. In particular embodiments, resistor 1 may be about 50 kohm, resistor 2 may be about 50 kohm, and resistor 3 (in some embodiments, the ground leg) may be about 10 kohm, yielding an equivalent functional resistance of about Mohm. In specific embodiments, the use of a transistor in place of a resistor is enabled by biasing and driving the transistor. In other embodiments, gain stages may be used to approximate a similar effect as the T-network. In certain embodiments, high sensitivity and efficiency detectors may be used, however these typically have increased surface area, expense, and significantly lower efficiency. For example, to achieve a voltage of at least 0.1 V (which is quite high from most photodiodes and may in some embodiments correspond to 0.1 microamp) without the embodiment of the apparatus disclosed herein, a single resistor gain of 1M would be required. In contrast, using the embodiment described, a gain of 100M is achieved that generates about 1-2 V based on only about 10 nanoamps, equating to at least three orders of magnitude in increased efficiency or measurement sensitivity.

Moreover, the use of the disclosures described herein makes certain embodiments recalcitrant to variability of the components used. In some embodiments with a large single resistor (for example 50 Mohm), the variability of that resistor becomes material in absolute terms. For example, increased accuracy components may be required to maintain accuracy, such as laser-trimmed or high precision resistors, for example with variability less than about 0.05%, 0.01%, 0.005%, 0.001% or less. In some embodiments, the optical detection apparatus uses components with a variability of about 1% or greater. In certain embodiments, the photodiodes of the apparatus are ground-referenced, which allows additional positive range of detection when compared to a voltage reference bias system, which is typically used because of the noise near the baseline (ground) reference in most systems. This is unnecessary in embodiments of the current disclosure. In other embodiments, the apparatus does not require a biased photodiode system due to its low noise and high sensitivity, achievable due to the inventions disclosed herein. For example in certain embodiments, the apparatus could use an avalanche photodiode, which allows additional sensitivity to low signal. Unfortunately, a bias system (including an avalanche bias) makes measuring quantity of signal difficult. In contrast, certain embodiments of the apparatus comprise photodiodes that are PIN diodes, feasible due to the disclosures herein, and resulting in a system which can quantitatively discriminate between two or more signal states, enabling the use of such a design to detect the signals of an exponential amplification reaction. Additionally, the use of these disclosures improves thermal stability of the components in some embodiments. For example, in a resistor for a given ppm/° C. of temperature resistance, resistance at a particular temperature may be approximated as R0+ΔT*ppm/10∧6. The use of smaller resistances in a more efficient design decreases the apparatus's sensitivity to thermal variation without the need to rely on higher precision components as discussed elsewhere herein. The increased efficiency enabled by these disclosures allow for reliable, low-cost, high SNR optical detection of nucleic acid amplification.

Optical Components

In some embodiments, the apparatus includes optical components for one or more detection channels, including for example, optical filters. In certain embodiments, the source illumination and detectors may be arranged in either colinear or orthogonal arrangements. In particular embodiments, the filters are high pass, low pass, band pass, or absorption filters. In some embodiments the filters are glass, or acrylic, or other materials that are transparent to visible light (or UV) and do not materially shift the frequency of light as it passes through the media. In some embodiments, the filters may be thin film (vapor deposited), doped into materials, or other techniques to achieve filtration of light that will be apparent to a practitioner in the art. Although many thin film filters specify a narrow capture angle, in some embodiments this may be overcome with high intensity, high degree of scattering and close proximity in conjunction with other embodiments described elsewhere herein, for example superpulsing. For example, in some embodiments, a LED with a rated specification of, for example, 350 mA could produce a luminous flux of about 10 lm, 25 lm, 50 lm, 75 lm, 100 lm, 125 lm, 150 lm, 175 lm, 200 lm or more. In embodiments which use a superpulse, the luminous flux of such an LED could exceed, for example, about 10, 20, 30, 40, 50 or more times that achievable using the rated capacity. Further, in some embodiments, the illumination distance between the excited fluorophore and the detector is less than about 10 mm, 8 mm, 7 mm, 6 mm, 5 mm, 4 mm, 3 mm, 2 mm, 1 mm, or less. The combination of very high luminous flux and a short light path to the detector allows certain embodiments, including those incorporating other aspects of disclosures herein, of achieving a directly detectable fluorescence signal with a high signal to noise ratio without the use of traditional optical components, for example dichroic mirrors, collimating lenses, radiometrically calibrated hardware components, expensive and high precision components, tight thermal stability control, as is described elsewhere herein and will be apparent to the practitioner. In some embodiments, the signal to noise ratio (SNR) of the optical system is less than about 1%, 0.01%, 0.005%, 0.0025%, 0.0005%, 0.00025%, 0.0001%, or less

In certain embodiments, the filters may be used in combination to exclude or attenuate certain wavelength ranges in favor of other ranges. In particular embodiments, the filters may operate to modify the ranges of light sources (for example, LEDs) or light detectors (for example, photodiodes). In certain embodiments, the light source may be white light, or may be of a particular wavelength range. This range may optionally be filtered to predominantly a subrange of the light. Some embodiments may include one or more color wheels, or multiple zone coating on the glass of a single detector or number of detectors.

In certain embodiments, the number of detector channels may equal the number of light channels, while in others they may differ. For instance, a single light source may excite multiple fluorescent dye targets of interest, but the emission spectra of those targets may differ in a manner distinguishable by the filters of their respective detectors. In specific embodiments, a two-channel optical detector may be constructed by using two light sources and two detectors. For example, in a particular embodiment, one channel may comprise a short pass coated glass/thin film filter on the light source (for example, a 500SP filter) and both an absorptive and coated glass/thin film on the detector side (for example, a 610SP on the glass, and a 525LP on the absorptive filter). In this embodiment, a second channel may comprise a coated glass/thin film on the light source (for example, a 640LP filter) and an absorptive filter (for example, 660LP) on the second channel detector side.

In some embodiments, the lenses may be held in a scaffold or filter holder, which may be a polymer or other plastic, metal or substance that preferably has a glassification temperature (T g) above that of local environment and does not allow the transmission of light through the material between the light source and the detector at the distances and intensities used the apparatus, for example in close proximity to the amplification chamber, for example ABS plastic, moldable polycarbonate, nylon and other materials that will be apparent to the practitioner. Such scaffold may be 3D printed, machined, injection molded, or formed in a variety of ways that will be apparent to the practitioner. In some embodiments, the use of adhesives, such as epoxy, is obviated by a compression fit. Such compression fit may be achieved in some embodiments by a filter holder tolerance of, for example, less than about 1 mm, 0.5 mm, 0.4 mm, 0.2 mm, 0.1 mm or less and an insertion force of less than about 5 lbs, 4 lbs, 3 lbs, 2 lbs, 1 lb or less. In embodiments with multiple optical elements, it may be advantageous to size the optical filters such that each optical element only fits in its designated aperture in the filter holder to prevent inadvertent misplacing of components.

In particular embodiments, the light source is a laser or an LED. In certain embodiments, one or more light sources may create the illumination for all of the detector channels. In specific embodiments, the light source or sources may be UV light. A short pass filter may incorporated which blocks UV light. In certain embodiments, a laser may use a collimating lens and constant current source. In other embodiments, laser scatter is used to illuminate a fluid in an orthogonal detection apparatus.

Control Systems

In some embodiments, the apparatus contains a printed circuit board, for example a flexible, rigid, or flex-rigid board, which may contain various electronic components for operation of the device. These components may include various sensors, for example, Hall effect sensors (for example for detecting the position of the piston, various openings in the apparatus, the position of indexer or other moving components within the apparatus), thermistors or thermocouples (for example for detecting the temperature of the amplification chamber in various locations, for detecting ambient temperature, for detecting the temperature of various components of the apparatus such as those in proximity to temperature-sensitive or temperature affected components, including those components whose activity is modulated by temperature such as resistors, photodiodes and the like), accelerometers (for example for detecting position, motion during operation, drops, stability and the like), light sensors (for example for detecting if various openings of the apparatus are open, for adjusting to differential light levels for example for modulating LCD displays or LEDs, for use with sample detection, optical detection and the like), current and voltage sensors (for example for detecting battery life, current draw, sample detection and other parameters for the function of the apparatus), button sensors (for example for determining the action or duration of a button press), position sensors (for example for determining cartridge index or proper alignment), RFID, NFC or other similar sensors (for example for determining component identification, compatibility, or instructions), and other sensors which will be apparent to the practitioner.

Functionality

In some embodiments, the apparatus is battery-powered and does not require connection to an external power source for charging. In particular aspects, the batteries are removable or disposable. In specific aspects, the entire apparatus is fully disposable. In certain embodiments, the apparatus is self-stable at room temperature for at least about 1 month, 3 months, 6 months, 9 months, 1 year, or more. In particular embodiments, the apparatus is capable of providing sample-to-result real-time PCR and RT-PCR, performing all the required steps for such reactions on the device without manual steps. In particular embodiments, the apparatus performs RT-PCR including sample collection, sample preparation, amplification, and detection. In certain embodiments, the apparatus reports a human-readable result to the user. In other embodiments, the apparatus reports a numeric or alphanumeric coded result to the user. In particular embodiments, the user is blinded to the interpretation of the code.

In certain embodiments, the apparatus performs sample-to-result PCR or RT-PCR and requires only one or more button-presses from a single button-press to operate. In particular embodiments, the entire process of sample-to-result PCR or RT-PCR is less than about 75 minutes, 60 minutes, 50 minutes, 45 minutes, 40 minutes, 35 minutes, 30 minutes, 25 minutes, 23 minutes, 20 minutes, 18 minutes, 15 minutes, 12 minutes, 10 minutes, or less. In particular embodiments, the apparatus incorporates a “zero-power state latch”, for example a momentary button that operates in a zero-power (or near-zero-power state, for example in some embodiments, less than about 1 μA, 500 nA, 250 nA, 150 nA, 100 nA, 50 nA, 25 nA, 10 nA, nA, 1 nA, or less) state until depressed, and then serves as a latch to maintain power during the operation of the apparatus until released by the microprocessor, at which time it returns to a zero-power state. In particular embodiments, the button is connected to both a contact in continuity with the battery as well as a microcontroller pin, such that on pressing the button, a P-FET (which is normally off) is pulled down to allow current from the battery. After a hold time shorter than perceivable by a user (for example, less than about 100 ms, 50 ms, 25 ms, 20 ms, 15 ms, 10 ms, 5 ms, 1 ms or less), the transistor is powered by the microprocessor and the apparatus remains powered. Upon release by the microprocessor, the P-FET closes, and the apparatus returns to a zero-power state.

In particular embodiments, the apparatus performs multiplex PCR. In particular embodiments, the apparatus is a sample-to-result disposable real-time PCR or RT-PCR device capable of detection sensitivity of less than about 5000 copies/mL, 4000 copies/mL, 3000 copies/mL, 2000 copies/mL, 1000 copies/mL, 500 copies/mL, 250 copies/mL, 200 copies/mL, 150 copies/mL, 100 copies/mL, 50 copies/mL, 25 copies/mL, 10 copies/mL, 1 copies/mL, or less.

In some embodiments, the apparatus has a mass of less than about 1 pound, 15 oz, 14 oz, 13 oz, 12 oz, 11 oz, 10 oz, 9 oz, 8 oz, 7 oz, 6 oz, 5 oz, 4 oz, 3 oz, 2 oz, or 1 oz. In certain aspects, the device is able to communicate wirelessly with a computer, cellular, or mesh network. In other aspects, the device is able to operate without any connection to a network.

FIG. 9 provides an exploded view of primary internal components of apparatus 50.

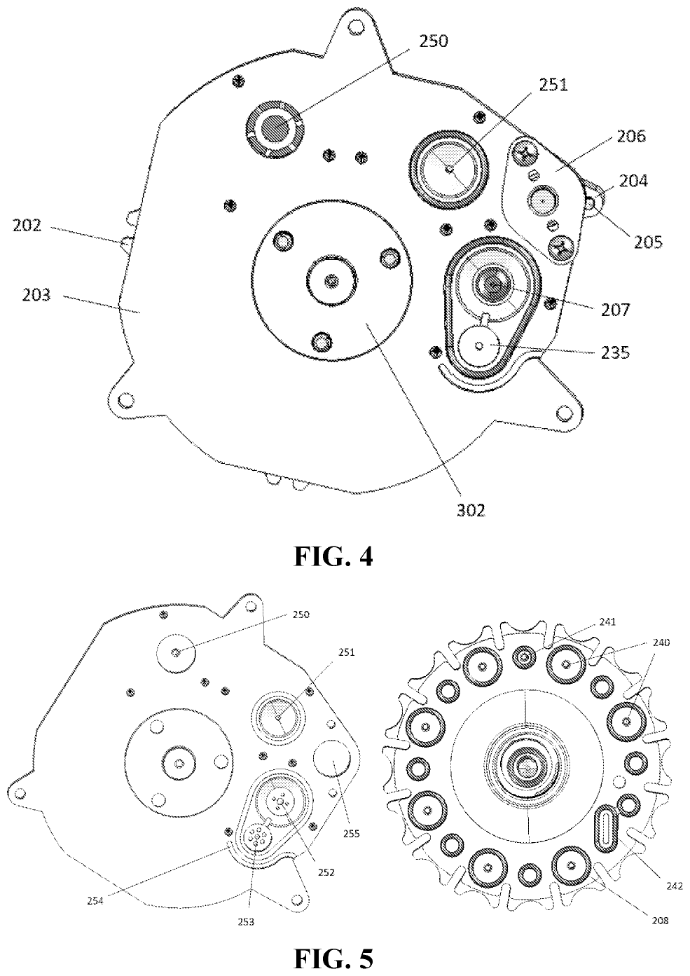

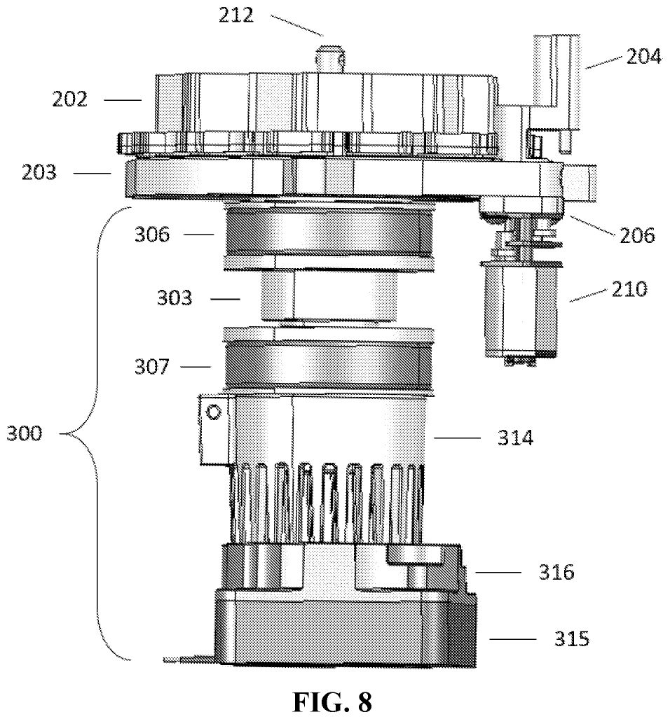

A legend of the reference numbers and description for components shown in FIGS. 1-9 is provided below. While some reference numbers are not discussed in the description of figures provided above, the function of such components is apparent in the descriptions below.

### FIGURE LEGEND FIGS. 1-10

100 apparatus housing
101 amplification PCB
102 interface PCB
103 display screen
104 momentary button
200 sample preparation module
201 prefilter
202 reagent cartridge
203 valve body
204 indexer
205 indexer magnet
206 indexer mount
207 check valve
208 O-rings or unitary O-ring
209 second syringe assembly
210 motor
211 motor shaft
212 valve stem
213 compression spring
214 lever arm
215 piercer
216 syringe O-ring
217 syringe housing
218 syringe gearbox
219 syringe lead screw
220 motor lead screw
221 filter
222 syringe plunger
232 indexer motor
233 first syringe assembly
234 piercer assembly
235 filter
240 piercer chamber aperture
241 piercer lever aperture
242 transfer O-ring
250 valve body piercer aperture
251 valve body first syringe aperture
252 valve body check valve aperture
253 valve body filter aperture
254 valve body heater groove
255 indexer aperture
260 valve body transfer aperture
261 valve body chamber holding
262 valve body chamber ingress
300 amplification module
301 hot end bistability magnets
302 hot end of amplification chamber
303 thermal break
304 cold end of amplification chamber
306 hot end heater
307 cold end heater
308 amplification chamber O-rings
309 cold end bistability magnets
310 piston body
311 piston cap
312 piston magnet
313 lyophilized reagents
314 cold end heat sink
315 fan
316 fan holder
400 detection module
401 optical housing
402 transparent window
403 optical scaffold
404 filter assembly
405 optical PCB

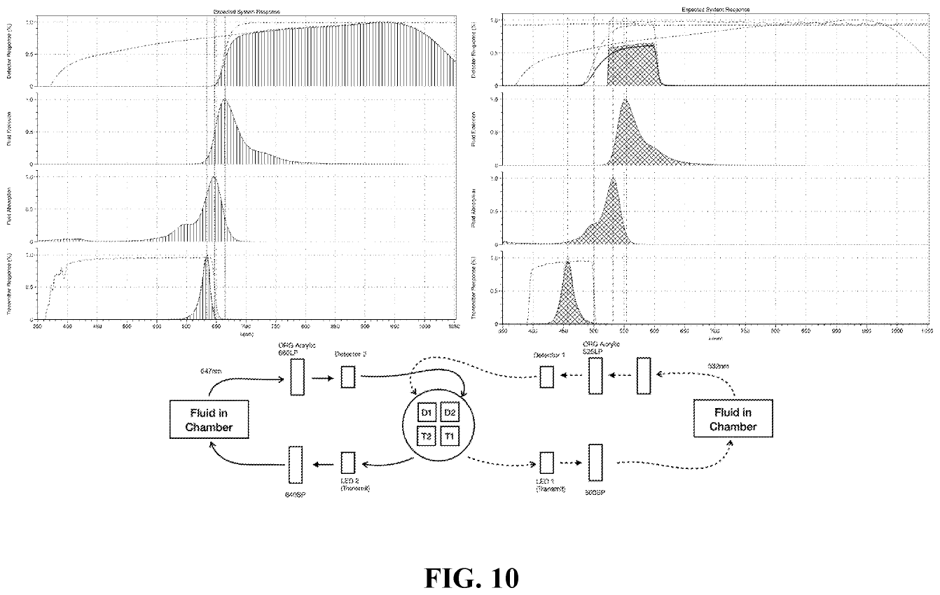

FIG. 10 provides a schematic and graphical overview of the optical system of apparatus 50. While certain components in the figure may be identified with specific part numbers, it is understood that embodiments of the present disclosure may include other comparable components than those specifically identified in the figure.

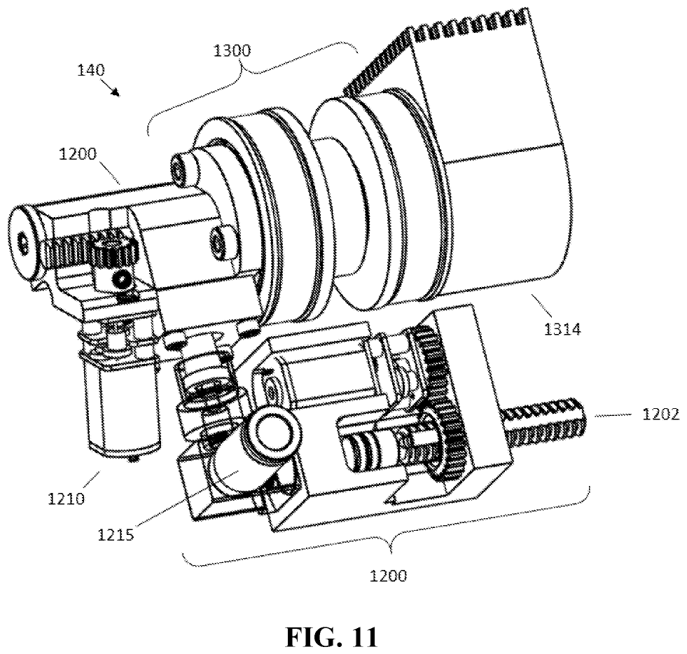

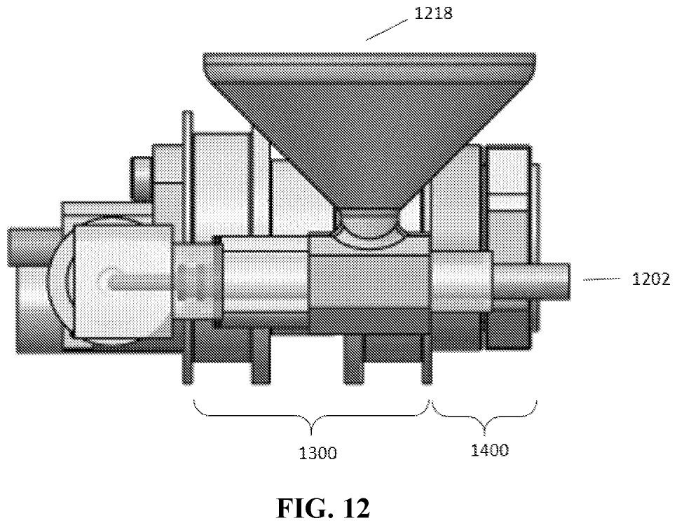

Referring now to FIGS. 11-12, an apparatus 140 for performing molecular diagnostics is shown in perspective view. As discussed further below, a user may provide a sample into receptacle 1218 (e.g. sometimes configured as a spittoon) and activate sample preparation module 1200 in apparatus 140 and begin the molecular diagnostic process.

Diluent

In certain embodiments, the apparatus contains a diluent. In particular embodiments, the diluent dilutes the raw biological sample. In other embodiments, the diluent dilutes a processed biological sample, which may be processed by the apparatus or using a separate process or device. In certain embodiments, the processed biological sample has been filtered or prefiltered as described elsewhere herein. In particular embodiments, the biological sample is passed through a mesh or net to exclude or partially exclude undesirable liquid fractions or solid materials from the biological sample to produce a filtrate.

In some embodiments, the diluent may contain one or more salts, buffers, nucleic acid stabilizers, polyol compounds, detergents, solvents or other organic compounds, proteins, peptides or animal products, oils, chelators, excipients, preservatives, nuclease inhibitors, or protease inhibitors. In certain embodiments, the diluent may contain oligonucleotide primers or probes, NTPs, or reducing agents.

In particular embodiments, the salts include manganese (Mn), magnesium (Mg), potassium (K), or ammonium. In specific embodiments, the manganese concentration may be about 0 mM, 0.35 mM, 0.4 mM, 0.45 mM, 0.5 mM, 0.55 mM, 0.6 mM, 0.65 mM, 0.7 mM, 0.75 mM, 0.8 mM, 0.85 mM, 0.9 mM, 0.95 mM, 1 mM, 1.05 mM, 1.1 mM, 1.15 mM, 1.2 mM, 1.25 mM, 1.3 mM, 1.35 mM, 1.4 mM, 1.45 mM, 1.5 mM, 2 mM, 3 mM, 4 mM, 5 mM, 6 mM, 7 mM, 8 mM, 9 mM, 10 mM, 11 mM, 12 mM, 13 mM, 14 mM, 15 mM. In certain embodiments, the magnesium concentration may be about 0.25 mM, 0.5 mM, 0.75 mM, 1 mM, 1.25 mM, 1.5 mM, 1.75 mM, 2 mM, 2.25 mM, 2.5 mM, 2.75 mM, 3 mM, 3.25 mM, 3.5 mM, 3.75 mM, or 4 mM, 5 mM, 6 mM, 7 mM, 8 mM, 9 mM, 10 mM, 11 mM, 12 mM, 13 mM, 14 mM, or 15 mM. In some embodiments the anion may be a chloride ion or acetate ion.

As will be understood to practitioners in the art, ions such as magnesium and potassium may both have a stabilizing effect on amplification reactions and thus the total concentration of magnesium may be decreased in embodiments incorporating a potassium chloride buffer (KCl). Conversely, in some embodiments, where an ammonium sulfate ((NH₄)₂SO₄) buffer is used in conjunction with, or in place of, a KCl buffer, due to the antagonistic effects between ammonium and magnesium ions in solution (and in some cases the lower KCl concentration), the magnesium concentration may be varied to a greater extent without compromising primer specificity.

In some embodiments, the concentration of ammonium sulfate may be about 0 mM. 0.25 mM, 0.5 mM, 0.75 mM, 1 mM, 1.25 mM, 1.35 mM, 1.45 mM, 1.5 mM, 1.55 mM, 1.65 mM, 1.75 mM, 2 mM, 2.25 mM 2.5 mM, 2.75 mM, 3 mM 3.5 mM, 4 mM, or higher. In other embodiments, the concentration of potassium may be about 20 mM, 25 mM, 30 mM, 35 mM, mM, 45 mM, 50 mM, 55 mM, 60 mM, 65 mM, 70 mM, 75 mM, 80 mM, 85 mM, 90 mM, mM, 100 mM, 105 mM, 110 mM, 115 mM, 120 mM, 125 mM, 130 mM, 135 mM, 140 mM, 145 mM, 150 mM, or more. In other embodiments, potassium acetate may also be used, as will be apparent to the practitioner.

In particular embodiments, the buffer may be Tris, HEPES, TEA, MOPS, sodium bicarbonate, or a combination thereof. In particular embodiments, the Tris HCl buffer may be at a concentration of about 5 mM, 10 mM, 15 mM, 10 mM, 25 mM, 50 mM, 75 mM, 100 mM, 150 mM, 200 mM, 225 mM, 250 mM, 300 mM or higher. In some embodiments, the pH may be about 6.5, 7, 7.5, 8, 8.5, or higher. In some embodiments, the pH may be about 6.5, 7, 7.5, 8, 8.2, 8.5, or higher. In other embodiments, it may be advantageous to use a buffer containing about 15 mM, 20 mM, 25 mM, 30 mM, 35 mM, 40 mM, 45 mM, 50 mM, 55 mM, 60 mM, 70 mM, or 75 mM bicine, tricine, TAPSO, MES (2-(N-morpholino)ethanesulfonic acid), as they may widen the permissible ranges of concentration of manganese for amplification reactions, for example, reactions using Tth DNA Polymerase.

In other embodiments, the diluent may contain one or more nucleic acid stabilizing compounds, such as spermidine (for example, see Frugier, M., “Synthetic polyamines stimulate in vitro transcription by T7 RNA polymerase.” Nucleic Acids Research, 1994., incorporated herein by reference), which may serve to stabilize nucleic acids, for example RNA to delay their degradation. In certain embodiments, the incorporation of spermidine may increase sensitivity by 0.5-1 Ct or more, depending on whether the sample is from a subject with a high endogenous RNase concentration or not.

In certain embodiments, the use of polyol compounds, such as glycerol may improve stability and sensitivity. In particular embodiments, the polyol compound is a glycerol. In specific embodiments, the glycerol may be at a concentration of about 1%, 2%, 3%, 4%, 5%, 6%, 7%, 8%, 9%, 10%, 11%, 12%, 15%, 16%, 17%, 18%, 19%, 20%, or higher. Similarly, in certain embodiments, the use of formamide (methanamide) may lower the melting temperature of nucleic acid duplexes. In particular embodiments, the formamide is deionized and may be included at about 0.5%, 0.75%, 1%, 1.25%, 1.5%, 2%, 3%, 4%, 5%, 6%, 7%, 8%, 9%, 10%, 11%, 12%, or higher.

In some embodiments, the use of a detergent, particularly a non-ionic detergent or surfactant may be advantageous, for example, about 0.0005%, 0.001%, 0.002%, 0.003%, 0.005%, 0.006%, 0.007%, 0.008%, 0.009%, 0.01%, 0.011%, 0.012%, 0.013%, or 0.015% Triton X-100, Tween, SDS, NP-40, sarkosyl (sodium lauroyl sarcosinate), Brij, CHAPS, octyl glucoside, or other chemicals that will be apparent to the practitioner. In certain embodiments, the diluent may contain a chelator, for example EDTA, EGTA, or other chemicals that will be apparent to the practitioner. In particular embodiments, the chelator will be EDTA at a concentration of about 0.01 mM, 0.05 mM, 0.1 mM, 0.5 mM, 0.75 mM, 1 mM, 2 mM, 4 mM, 6 mM, 8 mM, 10 mM, 12 mM, 14 mM, 16 mM, 18 mM, 20 mM, or higher. In other embodiments, the chelator may be included in the reaction mix as described elsewhere herein.

In particular embodiments, it may be advantageous to include one or more solvents, for example an organic solvent such as dimethyl sulfoxide (DMSO). This may be particularly useful when secondary or tertiary structures are likely to form in the DNA template, or when higher than typical GC content ratios (for example greater than about 52%) are present in the DNA template. In specific embodiments, DMSO may be included at about 0.1%, 0.5%, 1%, 2%, 3%, 4%, 5%, 6%, 7%, 8%, 9%, 10%, 11%, 12%, or higher. In other embodiments, alternative reagents such as betaine (N,N,N-trimethylglycine) may be included to aid amplification of high-GC content target. In particular embodiments, it may be included at a concentration of about 0.5M, 1M, 1.5M, 2M, 2.5M, 3M, 3.5M, 4M, or higher. Other PCR additives that may be useful in some embodiments include ethylene glycol and 1,2-propanediol, for example at concentrations of about 0.75M, 0.8M, 0.95M, 1M, 1.05M, 1.25M for either compound.

In other embodiments, the inclusion of a protein or animal product, such as bovine serum albumin (BSA) or gelatin (e.g. hydrolyzed collagen) may be advantageous, particularly with biological samples which tend to have a higher load of amplification inhibitors, including nucleases. In specific embodiments, BSA may be included in concentrations of about 5 μg/mL, 10 μg/mL, 20 μg/mL, 30 μg/mL, 40 μg/mL, 50 μg/mL, 60 μg/mL, 70 μg/mL, 80 μg/mL, 90 μg/mL, 100 μg/mL, 110 μg/mL, 120 μg/mL, 0.5 mg/mL, 1 mg/mL, 5 mg/mL or higher. In other embodiments, about 0.005%, 0.075%, 0.01%, 0.0125%, 0.015% gelatin or higher may be included.

In particular embodiments, an oil may be included to minimize evaporation, or coat components of the apparatus, for example in diagnostic kits. As many amplification reactions (for example, PCR) are done at elevated temperatures, the inclusion of an oil, such as a mineral oil which may float on top of the reaction mix can help to inhibit evaporation and perform an insulation function to keep heat in the reaction mix. In some embodiments, the oil is a mineral oil, for example medium and low viscosity mineral hydrocarbons or other oils and waxes, as well as mixes of paraffinic and naphthenic liquid hydrocarbons. In particular embodiments, the boiling temperature of the oil will be above about 100° C., 120° C., 150° C., 175° C., 200° C., or higher. In certain embodiments, the oil may be encapsulated or adsorbed onto a surface, or within a porous structure.

In some embodiments, the addition of compounds to the diluent which tend to stabilize reaction mix components is advantageous, particularly when the diluent is resuspending a reaction mix in dried or lyophilized format. For example, the inclusion of excipients may be helpful in certain embodiments. In specific embodiments, the excipients may be trehalose, polyethylene glycol (PEG), mannitol, sucrose, sorbitol, glycerol, or other reagents that will be apparent to the practitioner. In particular embodiments, the trehalose concentration may be about 1%, 2%, 3%, 4%, 5%, 6%, 7%, 8%, 10%, 12%, 15%, 18%, 20% or more. In some embodiments, the mannitol concentration may be about 0.1%, 0.5%, 1%, 1.5%, 2% or more. In certain embodiments, the PEG concentration may be about 1%, 2%, 3%, 4%, 5%, 6%, 7%, 8%, 10%, 12%, 15%, 18% or more. In selecting the correct concentration of trehalose, mannitol and PEG, it will be apparent to the practitioner to create a matrix of two or more reaction mix components, and via a design of experiment, select the optimal concentration by selecting the minimum Ct value conditions that do not adversely affect (by more than 5-10%, for example) the fluorescence delta data between baseline and amplified states.

Other embodiments that may be advantageous include those with nuclease inhibitors, for example, RNase inhibitors and DNase inhibitors. For example, some embodiments may include Poly(vinylsulfonic acid, sodium salt) (PVSA), ribonuclease inhibitor protein, anti-RNase antibodies, or other inhibitors that will be apparent to the practitioner.

In other embodiments, the reaction may include a ribo-oligonucleotides that acts as decoys to protect the target RNA (viral RNA). Active ribonucleases would primarily digest the ribo-oligonucleotides rather than the target RNA, thereby increasing the half-life of the target RNA. The ribo-oligonucleotides may comprise RNA isolated from organisms such as bacteria or yeast. In certain embodiments, these may also be chemically synthesized. In other embodiments, the ribo-oligonucleotides might be modified with modified nucleotides to confer greater resistance to ribonuclease digestion. Such modifications may include a thioester or a 2′-fluoro or a 2′-O-methyl.

In some embodiments, the diluent may comprise one or more preservatives, for example sodium azide, ProClin, microcide, 2-methylisothiazolinone, 2-chloroacetamide, antibiotics (e.g. gentamicin, ampicillin), sodium cacodylate, thimerosal, phenol, and other preservatives that will be apparent to the practitioner.

In particular embodiments, the diluent is stable at room temperature for at least about 7, 14, 21, 30, 45, 60, 90, 180, 365 days or more.

Reaction Mix

In certain embodiments, the apparatus or kit may comprise a reaction mix that contains one or more reagents. In particular embodiments, those reagents may include one or more polymerase enzymes, nuclease inhibitors, buffers, salts, oligonucleotide primers or probes, dNTPs, proteins, reducing agents, internal positive controls (IPC), excipients, or other amplification reactions as will be apparent to the practitioner.

In particular embodiments, it is advantageous to incorporate only those reagents into the reaction mix which may be stored in dried or lyophilized format, for example, in a bead, sphere, cake, or pellet format, or other formats that will be apparent to the practitioner. In this case, the reaction mix may be stored at room temperature for various time periods as described elsewhere herein.

In some embodiments, the polymerase enzymes may comprise a thermostable DNA polymerase with reverse transcriptase activity, for example a Tth enzyme. In other embodiments, the polymerase enzymes may comprise a DNA polymerase, for example a Taq enzyme. One or more enzymes may be used in combination. In particular embodiments, the concentration of the Tth may be about 0.0125 U/μl to about 1.25 U/μl, or about 0.025 U/μl to about 0.625 U/μl or about 0.05 U/μl to about 0.30 U/μl or about 0.1 U/μl to about 0.15 U/μl, or more particularly 0.125 U/μl. In particular embodiments, the concentration of the Taq may be about 0.01 U/μl to about 1.0 U/μl, or about 0.02 U/μl to about 0.5 U/μl or about 0.04 U/μl to about 0.25 U/μl or about 0.08 U/μl to about 0.125 U/μl, or more particularly 0.10 U/μl. In other embodiments, the enzymes in the reaction mix may be inactivated with a hotstart antibody, aptamer, or chemical inactivation as will be apparent to the practitioner. In some embodiments, a heat-stable reverse transcriptase enzyme may be combined with a thermostable DNA polymerase. In particular embodiments, the heat-stable reverse transcriptase may be, for example, NxtScript 2G or 3G (Roche) and the thermostable DNA polymerase KAPA3G (Roche).

In other embodiments, the nuclease inhibitors may include RNase inhibitors or DNase inhibitors. Examples of nuclease inhibitors include RNA oligonucleotides, modified ribo-oligonucleotides, and total RNA.

In some embodiments, a reducing agent is included in the reaction mix. The reducing agent may be dithiothreitol (DTT) or tris(2-carboxyethyl)phosphine) (TCEP), or may include other reagents that will be apparent to the practitioner, such as N-acetyl cystine, glutathione, or various free radical scavengers, including for example glutathione, dithiobutylamine and cysteamine. In specific embodiments, the oxidation of various reducing agents, for example, DTT, may be minimized by avoiding exposure to air, for example by storing and packaging using nitrogen, argon, sulfur hexafluoride, or other inert gases that will be apparent to the practitioner.

In certain embodiments, the DTT concentration may be about 0.3 mM, 0.4 mM 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 10.5 mM, 11 mM, 11.5 mM, 12 mM, 15 mM, 20 mM, or higher. In particular embodiments, the concentration of TCEP may be about 0.3 mM, 0.4 mM 0.5 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 10.5 mM, 11 mM, 11.5 mM, 12 mM, 20 mM or higher.

In various embodiments, additional stabilizers such as BSA, excipients, or spermidine (as described elsewhere herein) may be added to the reaction mix to improve performance. In certain embodiments, elements of the buffers, salts, oligonucleotide primers and probes may be included in the reaction mix. In specific embodiments, one or more of the primers may be placed into the diluent, while others are in the reaction mix to mitigate the risk of spurious or non-specific amplification during the manufacturing process owing to reagents existing at various temperature ranges other than those for which the assay was optimized. In specific embodiments, the dNTP concentration may be about 100 nM, 125 nM, 150 nM, 175 nM, 200 nM, 225 nM, 250 nM, 275 nM, 300 nM, 325 nM, 350 nM, 375 nM, 400 nM, 425 nM, 450 nM, 475 nM, 500 nM or more.

Direct Lysis

There are several challenges for apparatuses and kits which use a direct lysis approach to sample preparation (in contrast, for example, to an extraction-based approach), particularly when the biological sample matrix is saliva, and particularly when the nucleic acid of interest is RNA. Because of the presence of various nucleases in saliva and the fragility of RNA relative to DNA, as well as the high degree of variability in the activity of RNases and other inhibitory compounds among different subjects, as well as within a given subject depending on hydration status, time of day, and many other factors, producing consistent, repeatable, reliable results using direct lysis with RNA targets (for example, viral RNA targets such as COVID-19) may be difficult.

However, in some embodiments, the combination of several independent processes disclosed herein is able to produce reliable detection of RNA targets using direct lysis of saliva with improved performance and low variability. In particular, some embodiments comprise 1) using a prefilter to remove or reduce liquid fractions or solid components of a biological sample such as saliva (for example, as described herein), 2) diluting the biological sample, 3) rapidly changing the temperature of the biological sample by mixing the biological sample with a reaction mix including a Tth enzyme, and 4) optionally including a reducing agent, which is not essential for all saliva samples, but for subjects with a higher activity of RNases and other nucleases, can be a benefit to reducing variability.

In certain embodiments, the reducing agent (for example, TCEP) is added to the biological sample (for example, raw saliva), which is then heated to about 94C (with specific embodiments at above about 60C, 65C, 70C, 75C, 80C, 85C, 90C, 95C, or 100C) for about 10 minutes (with specific embodiments at more than about 1 minute, 2 minutes, 3 minutes, 4 minutes, 5 minutes, 6 minutes, 7 minutes, 8 minutes, 9 minutes, or 10 minutes). In particular embodiments, depending on the biological sample used, a precipitate may form during this process. In certain embodiments, the precipitate may be reduced by the use of cotton fiber as a prefilter. In particular embodiments, the prefilter is made of a naturally derived material, such as cotton, cellulose, or wool fiber, including blends with synthetic fibers. In particular embodiments, the density of the prefilter is less than about 1.75 g/cm∧3, 1.5 g/cm∧3, 1.25 g/cm∧3, 1 g/cm∧3, or lower. In further embodiments, the apparatus may preferentially select the liquid, non-precipitate portion of the raw or filtered biological sample after reduction by the reducing agent.

In some embodiments, the biological sample is directly added to a reaction mix incubated at a lysis temperature such that the biological sample would be rapidly heated to the same temperature as the reaction mix. In particular embodiments, the reaction mix is overlaid with mineral oil or a wax. The reaction mix is comprised of a heat-stable reverse transcriptase and a thermostable DNA polymerase. In various embodiments, both enzyme activities are included in a single enzyme. In particular embodiments, the enzyme is Tth or modified Tth or related enzymes. In other embodiments, the reaction mix two different proteins embodying these activities. In various embodiments, the reaction mix includes a reducing agent.

In particular embodiments, the use of a Tth enzyme is native Tth or an enzyme that has been modified for particular traits (for example, HawkZ05 (Roche)). In certain embodiments, the Tth is a hotstart version (as described elsewhere herein). In certain embodiments, the Tth enzyme may be sourced from kits, including for example, PrimeDirect Probe RT-qPCR Mix, Cat #RR650A from Takara Bio Inc., MasterAmp Tth DNA Polymerase, Cat. TTH72250 from Lucigen Corp., or Tth DNA Polymerase Cat. 104005 and Cat 104025 from Boca Scientific.

In some embodiments, the biological sample is diluted about 2X, 3X, 4X, 5X, 6X, 7X, 8X, 9X, 10X, 12X or more. In other embodiments, the temperature of the reaction mix is above about 50° C., 55° C., 60° C., 65° C., 70° C., 75° C., 80° C., 85° C., 90° C., 92° C., 93° C., 94° C., 95° C., 96° C., 97° C., 98° C., 99° C., or higher. In particular embodiments, the mixture is raised to one or more higher temperatures for about, 30 seconds, 45 seconds, 1 minute, 90 seconds, 2 minutes, 3 minutes, 4 minutes, 5 minutes, 6 minutes, 7 minutes, 10 minutes, or longer. In other embodiments, the period between when the biological sample is mixed with the reaction mix and when it reaches the elevated temperature (as described herein) is less than about 1 minute, seconds, 45 seconds, 30 seconds, 25 seconds, 20 seconds, 15 seconds, 12 seconds, 10 seconds, 9 seconds, 8 seconds, 7 seconds, 6 seconds, 5 seconds, 4 seconds, 3 seconds, 2 seconds, or 1 second.

In some embodiments, a sample (e.g. saliva) is injected into a pre-heated buffer comprising a reducing agent and incubated for 1, 2, 3, 4, 5, 7, 10 minutes or longer. In particular embodiments, the incubation temperature is 70, 80, 85, 90, 95, 99C or higher. In specific embodiments, the reducing agent is DTT, TCEP, glutathione, or beta-mercaptoethanol and the concentration is 1, 2, 5, 10, 20 mM or higher. After incubation, the temperature can be decreased to a temperature compatible with a reverse transcriptase such as MMLV-RT or mutants of MMLV-RT such SuperScript or NxtScript. In some embodiments, the temperature is lowered to 40, 45, 50, 55, 60, 65C or higher. After reaching the lowered temperature, the heat-processed saliva can be combined with enzymes and other components required for RT-PCR such that reverse transcription and PCR is now enabled.

In specific embodiments, the diluent is combined with a reaction mix, which may optionally include a reducing agent or a control and which may be a liquid reaction mix or a dried or lyophilized reaction mix. After the mixing, the resuspended reaction mix is heated as described herein. After heating, the heated, resuspended reaction mix is mixed with a biological sample, preferably a prefiltered biological sample. The rapid temperature change of the biological sample deactivates the endogenous nucleases present in the sample by denaturing the proteins and aids in lysis of the sample. The inclusion of a Tth allows the entire reaction mix to be heated to the higher temperature without the destruction of reverse transcriptase enzymes which would otherwise be required in the reaction mix to reverse transcribe RNA targets. This simplifies the apparatus or kit necessary to support the chemistry as it avoids the need for separate chambers. The inclusion of a hotstart is also an embodiment that simplifies the apparatus or kit for some assays which might otherwise be subject to non-specific amplification as the reaction mix is heated through the lower temperature zones. Understanding that in general a simplified apparatus or kit is preferred, embodiments where more than one chamber may be used to separate components of the reaction mix, biological sample, diluent, etc., are also embodiments that are disclosed herein in light of the present invention.

In some embodiments, the apparatus may comprise a housing. In particular embodiments, the housing allows access for, or is contiguous with, a receptacle. FIG. 12 provides an external view of an embodiment of the apparatus, including an exemplary receptacle configured as a spittoon. In particular embodiments of the apparatus, a user may provide a biological sample, for example saliva, through the receptacle 1218, which may be located adjacent to a sample preparation dock, 1217, optionally containing a check valve 1216.

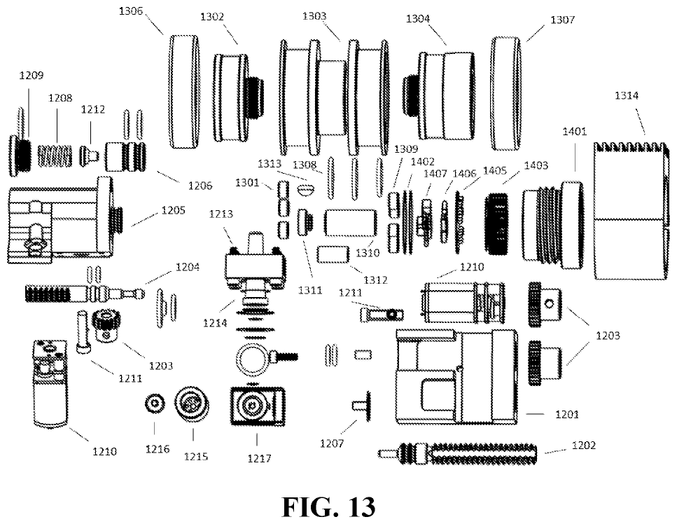

In particular embodiments, a motor 1210 disposed near a sample preparation housing 1201, may through the action of a motor shaft 1211, sample preparation gears 1203, and other such structures as will be apparent to the practitioner, move a sample preparation lead screw 1202 within the sample preparation housing 1201 to draw a biological sample in through the receptacle 1218 and the receptacle housing 1215 and receptacle check valve 1216. By reversing the direction of the sample preparation lead screw 1202, the biological sample may be advanced through a prefilter 1214, shown in FIG. 13 disposed within a prefilter housing 1213 with retrograde flow into the receptacle 1218 prevented or minimized by the function of the receptacle check valve 1216.

In certain embodiments, the diluent disposed in ingress housing 1205 may be moved into amplification chamber 1300, by the unsealing of the amplification module 1300, for example by the withdrawal of the valve stem 1204 in the presence of a vacuum or partial vacuum in the amplification module 1300, or may be assisted by gravity, or may be advanced by the action of the valve stem 1204, for example through the action of the sample preparation gear 1203 and motor 1210 and motor shaft 1211 associated with the ingress housing 1205 or advanced by the action of the diluent spring 1208 and the diluent nozzle 1212 to effect the passage of a diluent into the amplification module 1300.

After activation of one or more heaters in the amplification module 1300, for example, the hot end heater 1306 or the cold end heater 1307, or both, and having reached an elevated temperature as described elsewhere herein, the filtered biological sample, may be passed into the ingress housing 1205. This passage may be effected in a similar manner to the diluent, and the use of one or more check valves, or the interlocking action of the valve stem 1204 and the diluent plug 1206 may be used to prevent or mitigate retrograde flow from the amplification chamber 1300.

In particular embodiments, the amplification chamber 1300 may contain a piston comprised, for example of a piston body 1310, piston cap 1311, and piston magnet 1312. The filtered diluted biological sample may be amplified within the amplification chamber by methods and with reagents as described elsewhere herein, including for example, presence of a reagent mix in lyophilized format disposed within the amplification chamber 1300, for example, the lyophilized reagents 1313.

As described elsewhere herein, in some embodiments, the detection module 400 may detect the presence of an amplifying nucleic acid within the amplification chamber 300 by comparing baseline fluorescence levels to potentially amplifying fluorescence levels via the transparent window 1402 and the action of the LEDs 1406 and filters 1407 and photodetectors in the optical scaffold 1403 comprising the filter assembly and the optical PCB 1405 disposed in the optical housing 1401.

Kits

In particular embodiments, the present disclosure may comprise a kit, for example a kit wherein the reaction mix is disposed within one or more sealed containers, for example small format tubes appropriate for amplification on conventional amplification devices including PCR, examples of which will be apparent to the practitioner. In specific embodiments, the tubes may incorporate a small valve, for example a duckbill or similar check valve through which the liquid diluent may be added to a lyophilized reaction mix (such diluent optionally containing an oil as described herein) to resuspend the reaction mix. In certain embodiments, the resuspended reaction mix may be increased in temperature as described elsewhere herein, and having reached such temperature, again using the optional valve feature, but alternatively using a foil, plastic, or other membrane which is perforated during use relying on the oil to mitigate or prevent material evaporation at the elevated temperature, the biological sample may be added to the tube, for example, in dilutions and ratios described elsewhere herein. In other embodiments, the reaction mix will be in liquid form, including for example an oil as described herein, and may be stored at various temperatures for improved stability prior to use, for example about −80° C., −70° C., 20° C., 4° C., or room temperature.

### Figure Legend—FIGS. 11-13

1200 sample preparation module
1201 sample preparation housing
1202 sample preparation lead screw
1203 sample preparation gears
1204 valve stem
1205 ingress housing
1206 diluent plug
1207 check valve
1208 diluent spring
1209 diluent cap
1210 motor
1211 motor shaft
1212 diluent nozzle
1213 prefilter housing
1214 prefilter
1215 receptacle housing
1216 receptacle check valve
1217 sample preparation dock
1218 receptacle
1300 amplification module
1301 hot end bistability magnets
hot end of amplification
1302
chamber
1303 thermal break
cold end of amplification
1304
chamber
1306 hot end heater
1307 cold end heater
1308 amplification chamber O-rings
1309 cold end bistability magnets
1310 piston body
1311 piston cap
1312 piston magnet
1313 lyophilized reagents
1314 cold end heat sink
1400 detection module
1401 optical housing
1402 transparent window
1403 optical scaffold
1404 filter assembly
1405 optical PCB
1406 LEDs
1407 filters
   FIG. 13 provides a schematic and graphical overview of the optical system of apparatus 40. While certain components in the figure may be identified with specific part numbers, it is understood that embodiments of the present disclosure may include other comparable components than those specifically identified in the figure.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All examples disclosed herein are non-limiting examples. All such similar substitutes and modifications apparent to those skilled in the art are deemed to be within the spirit, scope and concept of the invention as defined by the appended claims.

### V. References

The following references, to the extent that they provide exemplary procedural or other details supplementary to those set forth herein, are specifically incorporated herein by reference.
U.S. Pat. No. 6,132,996
U.S. Pat. No. 6,453,242
U.S. Pat. No. 6,503,717
U.S. Pat. No. 6,551,838
U.S. Pat. No. 6,537,256
U.S. Pat. No. 6,534,261
U.S. Pat. No. 6,599,692
U.S. Pat. No. 6,689,558
U.S. Pat. No. 6,794,136
U.S. Pat. No. 7,030,215
U.S. Pat. No. 7,067,317
U.S. Pat. No. 7,070,934
U.S. Pat. No. 7,070,592
U.S. Pat. No. 7,253,273
U.S. Pat. No. 7,262,054
U.S. Pat. No. 7,361,635
U.S. Pat. No. 7,524,652
U.S. Pat. No. 7,888,015
U.S. Pat. No. 8,398,940
U.S. Pat. No. 8,640,555
U.S. Pat. No. 8,720,198
U.S. Pat. No. 9,044,755
U.S. Pat. No. 9,105,384
U.S. Pat. No. 9,427,739
U.S. Pat. No. 9,476,895
U.S. Pat. No. 9,519,000
U.S. Pat. No. 9,623,415
U.S. Pat. No. 9,932,634
U.S. patent Ser. No. 10/011,841
U.S. patent Ser. No. 10/017,807
U.S. patent Ser. No. 10/040,071
U.S. patent Ser. No. 10/112,196
U.S. patent Ser. No. 10/196,678
U.S. Pat. No. 8,795,965
U.S. Pat. No. 8,211,202
U.S. Patent App. No. 20100291536
U.S. Patent App. No. 20110059502
U.S. Patent Pub. No. 20180193834
U.S. Patent Pub. No. 20020068357
U.S. Patent Pub. No. 20040014117
U.S. Patent Pub. No. 20050013732
U.S. Patent Pub. No. 20050064474
U.S. Patent Pub. No. 20050267061
U.S. Patent Pub. No. 20060188987
U.S. Patent Pub. No. 20070218528
U.S. Patent Pub. No. 20100137152
U.S. Patent Pub. No. 20110312072
U.S. Patent Pub. No. 20110318728
U.S. Patent Pub. No. 20150079598
U.S. Patent Pub. No. 20150136602
U.S. Patent Pub. No. 20150273472
U.S. Patent Pub. No. 20160263579
U.S. Patent Pub. No. 20170074258
U.S. Patent Pub. No. 20170173588
U.S. Patent Pub. No. 20170247745
U.S. Patent Pub. No. 20170304829
U.S. Patent Pub. No. 20180154364
AU2015210344
CN107653300
WO2014113663
WO2018005870
Antoon L. et. al., Nucleic Acids Research, Vol. 40, No. 2 e10.2012.
Applied Biosystems™ by Thermo Fischer Scientific™ “Getting Started Guide,” 2015.
Barany F, Proc. Natl. Acad. Sci. 88:189-193, 1991.
Bengtsson et al., Nucleic Acids Res 31: e45, 2003.
Chia et al.; Journal of Mechanics, Vol. 27, Issue 03, pp. 357-364, Sept. 2011.
Choi et al.; Biosensors and Bioelectronics 115, 83-90, 2018.
Compton, J., Nature 350: 91-92, 1991.
Farrar et al.; Clinical Chemistry, 61 (1) 145-153, Jan 2015.
Fire A, Xu S-Q, Proc. Natl Acad. Sci. USA 92:4641-4645, 1995.
French et al., Mol Cell Probes 15:363-374, 2001.
Gootenberg etal., Science, 356(6336): 438-442, 2017.
H. J. Karlsson et al., Bioorganic & Medicinal Chemistry 11:1035-1040, 2003.
Haugland, Handbook of Fluorescent Probes and Research Chemicals, 6th ed. 1996.
Higuchi et al., Biotechnology 10:413-417, 1992.
Holland et al., Proc Natl Acad Sci USA 88:7276-7280, 1991.
Hoser M J, Mansukoski H K, Morrical S W, Eboigbodin K E, PLOS ONE 9: e112656, 2014.
http://tools.thermofisher.com/content/sfs/manuals/cms 041902.pdf
https://www.idtdna.com/pages/education/decoded/article/designing-per-primers-and-probes
https://www.idtdna.com/pages/products/custom-dna-rna/oligo-modifications/fluorophores/freedom-dyes
https://www.ncbi.nlm.nih.gov/probe/docs/projtaqman/
Ishiguro et al., Anal Biochem 229:207-213, 1995.
Jones, M. E., et al., Computational Biology Journal, Article ID 601504, 2015.
Kandimalla and Agrawal, Bioorg Med Chem 8:1911-1916, 2000.
Kaur etal., Biochemistry 45:7347-7355, 2006.
Kaur H, Arora A, Wengel J, Maiti S., Biochemistry 45:7347-7355, 2006.
Kutyavin Nucleic Acids Res 38: e29, 2010.
Kutyavin, Nucleic Acids Res. 41: e191, 2013.
Lee et al., Anal Chim Acta;457:61-70, 2002.
Legendre et al.; Anal Chem.;78(5):1444-51, 2006.
Li et al., Nucleic Acids Res 30: e5, 2002.
Liew, Clin Chem 50:1156-1164, 2004.
Liu et al.; Anal. Chem., 76, 1824-1831, 2004.
Monis et al., Anal Biochem 340:24-34, 2005.
Morrison et al., Anal Biochem 183:231-244, 1989.
Myhrvoid et al., Science, 360(6387), 444-448, 2018.
Nazarenko et al., Nucleic Acids Res 25:2516-2521, 1997.
Nazarenko et al., Nucleic Acids Res 30: e37, 2002.
Nazarenko I A, Bhatnagar SK, Hohman RJ, Nucleic Acids Res 25:2516-2521, 1997.
Nielsen et al., Science 254:1497-1500, 1991.
Niemz et al.; Trends Biotechnol., 29(5): 240-250, 2011.
Notomi T, et al., Nucleic Acids Res. 28: e63, 2000.
Pals et al., J Biochem Biophys Methods, 47:121-129, 2001.
Piepenburg O, Williams C H, Stemple D L; Armes N A, PLoS Biology 4: e204, 2006.
Pons et al., Chembiochem 7:1173-1176, 2006.
Prediger, Integrated Data Technologies, 2015.
Prediger, Integrated Data Technologies, 2018.
Prediger, Integrated DNA Technologies, 2013.
Richard P. Haugland, Handbook of Fluorescent Probes and Research Chemicals (6th ed. 1996).
Schneeberger et al., PCR Methods Appl 4:234-238, 1995.
Sherrill et al., J Am Chem Soc, 126:4550-4556, 2004
Tuma et al., Anal Biochem., 268:278-288, 1999.
Tyagi and Kramer, Nat Biotechnol 14: 303-8, 1996.
Vincent M, Xu Y, Kong H, EMBO Rep 5:795-800, 2004.
Walker G T, Fraiser M S, Schram J L, Little M C, Nadeau J G, Malinowski D P, Nucleic Acids Res 20:1691-1696, 1992.
Wang et al., Anal Biochem 356:303-305, 2006.
Whitcombe et al., Nat Biotechnol 17: 804-807, 1999.
Zanoli and Spoto, Biosensors 3(1): 18-43, 2013.
Zhang, et al.; Sensors and Actuators B: Chemical, 126924, 2019.
Zhao, S. and Fernald, R. D., J Comput. Biol., 12(8): 1047-1064, 2005.
Lorenz, TC, J Vis Exp., (63): e3998, 2012.
Roux, Cold Spring Harb Protoc., 2009
Robertson and Walsh-Weller, Methods Mol Biol., 98:121-54, 1998.
Zainabadi et al., PLoS One, 14(2): e0210813; 2019.
Kulinski et al., Biomed Microdevices, 11(3): 671-678, 2009.
Darbandi et al., Middle East Fertitility Society Journal, 23(3): 216-219, 2018.
Wu et al., Biotechniques, 58(6): 293-300, 2014.
Sildorova et al., Experimental Dermatology, 2011
Kajivara et al., J Biomol Tech, 26(4):118-124, 2015
Rutledge, R. G., Nucleic Acids Research, Vol. 32, No. 22, 2004.
Liu, W. and Saint, D. A., Biochem. Biophys. Res. Commun., 294,347-353, 2009.
NIST/SEMATECH e-Handbook of Statistical Methods, 2013 http://itl.nist.gov/div898/handbook/, incorporated herein by reference
Zhang G., et al., Macromolecules, 51 (5), pp 1927-1936, 2018.
Drouin et al, J. Polaina and A. P. MacCabe (eds.), Industrial Enzymes, 379-401, 2007.
Batista and Pacheco, Journal of Microbiological Methods, 152: 98-104, 2018.
Beerli et al. Nature Biotechnol. 20:135-141, 2002.
Pabo et al. Ann. Rev. Biochem. 70:313-340, 2001.
Isalan et al. Nature Biotechnol. 19:656-660, 2001.
Segal et al. Curr. Opin. Biotechnol. 12:632-637, 2001.
Choo et al. Curr. Opin. Struct. Biol. 10:411-416, 2000.
Gaj et al., Trends in Biotechnology 31(7), 397-405, 2013.
Bartlett JMS, Stirling D, Methods in Molecular Biology, 2003.
Kerry, Joseph; Butler, Paul, Wiley & Sons, 2008.
Haff and Smirnov, Nucl. Acids Res. 25:3749-50, 1997;
Xu et al., Anal. Chem. 69:3595-3602; 1997.
Sauer et al., Nucl. Acids Res. 31: e63, 2003.
Gootenberg et al., Science, 356(6336): 438-442, 2017
Myhrvoid et al., Science, 360(6387), 444-448, 2018.
de la Rica, Anal. Chem., 81, 10, 3830-3835, 2009.
Farrar et al., Molecular Diagnostics, 79-102, 2017.
Omar and MatJafri, Sensors, 9(10): 8311-8335, 2009.
Urusov et al., Biosensors, 9(3): 89; 2019.
Toehold Probes for Nucleic Acid Detection, New molecular probe can distinguish DNA and RNA sequences with unprecedented accuracy, Wyss Institute, 2012
Tyagi and Kramer, F1000 Med Rep, 4:10, 2012.
Nojima et al., Nucleic Acids Symposium Series, Volume 1, Issue 1, 105-06, 2001.
Fiala and Diamandis, BMC Medicine, Volume 17, Article number: 159, 2019.
Woods-Bouwens, et. al., Journal of Mol. Diagnostics., Vol. 19, No. 5., 2017.
Lee et al., Journal of Forensic Sciences, 56(s1): S179-S182, 2011.
Chen et al., Journal of the Formosan Medical Assoc, 118(1): 395-400, 2019.
Papa, Sports Med Arthrosc, 24(3): 108-115, 2016.
Rajapaksha et al., J Vet Med B Infect Dis Vet Public Health, 49(7): 322-4, 2002.
Ezer et al., Journal of Heredity, 87(6):450-5, 1996
Yoon et al., Asian-Australas J Anim Sci, 18(10), 2005.
Mokany, et al., J. Am. Chem. Soc. 2010, 132, 3, 1051-1059.

What is claimed:

1. An apparatus for nucleic acid amplification, the apparatus comprising:
a receptacle configured to receive a sample;
a heater;
an amplification chamber; and
a reaction mix, wherein:
the apparatus is configured to heat the reaction mix to a temperature of at least about and
the apparatus is configured to combine the sample with reaction mix after the reaction mix has been heated to the temperature of at least about 60° C.

2. The apparatus of claim 1 wherein the apparatus is configured to heat the reaction mix to a temperature of approximately 95° C.

3. The apparatus of claim 1 or 2 further comprising a diluent.

4. The apparatus of claim 3 wherein the apparatus is configured:
to heat the diluent and the reaction mix to a temperature of at least about 60° C.; and
the apparatus is configured to combine the sample with the diluent and the reaction mix after the diluent and the reaction mix has been heated to the temperature of at least about 60° C.

5. The apparatus of claim 4 wherein the apparatus is configured to heat the diluent and the reaction mix to a temperature of approximately 95° C.

6. The apparatus of any of claims 1-4 further comprising a prefilter.

7. The apparatus of any of claim 6 wherein the prefilter is configured to filter the sample after the sample is received in the receptacle and prior to the sample being introduced into the amplification chamber.

8. The apparatus of claim 6 wherein the prefilter is configured to mechanically filter the sample.

9. The apparatus of any of claims 1-8 wherein the reaction mix comprises a polymerase enzyme.

10. The apparatus of claim 9 wherein the polymerase enzyme is a thermostable DNA polymerase with reverse transcriptase activity.

11. The apparatus of claim 9 wherein the thermostable polymerase with reverse transcriptase activity is Tth polymerase.

12. The apparatus of claim 9 wherein the polymerase enzyme is a thermostable DNA polymerase.

13. The apparatus of claim 12 wherein the thermostable DNA polymerase is Taq polymerase.

14. The apparatus of any preceding claim wherein the reaction mix contains a reducing agent.

15. The apparatus of claim 14 wherein the reducing agent comprises dithiothreitol (DTT).

16. The apparatus of claim 14 wherein the reducing agent comprises tris(2-carboxyethyl)phosphine) (TCEP).

17. The apparatus of any of claims 1-17 wherein the apparatus is configured to cycle the sample and the reaction mix between a first temperature and a second temperature to amplify nucleic acids in the amplification chamber, wherein the first temperature is a lower temperature than the second temperature.

18. The apparatus of any preceding claim wherein the receptacle is configured as a spittoon.

19. A method of nucleic acid amplification, the method comprising:
filtering a raw biological sample to provide a filtered biological sample;
heating a reaction mix to a temperature of at least about 60° C. to provide a heated reaction mix; and
combining the filtered biological sample with the heated reaction mix.

20. The method of claim 1 wherein the reaction mix is heated to a temperature of approximately 95° C.

21. The method of claim 19 or claim 20 further comprising diluting the filtered biological sample prior to combining the filtered biological sample with the heated reaction mix.

22. The method of any of the preceding claims wherein filtering the raw biological sample comprises mechanically filtering the raw biological sample.

23. The method of any of the preceding claims wherein the reaction mix comprises a polymerase enzyme.

24. The method of claim 23 wherein the polymerase enzyme comprises a thermostable DNA polymerase with reverse transcriptase activity.

25. The method of claim 23 wherein the polymerase enzyme comprises a Tth enzyme.

26. The method of claim 23 wherein the polymerase enzyme comprises a DNA polymerase.

27. The method of claim 26 wherein the DNA polymerase comprises a Taq enzyme.

28. The method of any preceding claim wherein the reaction mix contains a reducing agent.

29. The method of claim 28 wherein the reducing agent comprises dithiothreitol (DTT).

30. The method of claim 28 wherein the reducing agent comprises tris(2-carboxyethyl)phosphine) (TCEP).

31. The method of any preceding claim further comprising cycling the filtered sample and the reaction mix between a first temperature and a second temperature to amplify nucleic acids in the filtered sample and reaction mix, wherein the first temperature is a lower temperature than the second temperature.

32. An apparatus for nucleic acid amplification, the apparatus comprising:
a mechanical prefilter;
a diluent;
a reaction mix; and
an amplification chamber, wherein:
the diluent and reaction mix are combined into a mixture and raised to a temperature of at least about 60° C.; and
a biological sample is combined with the mixture after it has reached at least about 60° C.

33. The apparatus of claim 32 where the reaction mix contains a reducing agent.

34. The apparatus of claim 32 or claim 33 where the reaction mix is lyophilized.


SEQUENCE LISTING (78 sequences)

SEQ ID NO: 1 - DNA, 243 bp, Severe acute respiratory syndrome coronavirus 2
tggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagaca

SEQ ID NO: 2 - DNA, 157 bp, Severe acute respiratory syndrome coronavirus 2
ttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctg

SEQ ID NO: 3 - DNA, 290 bp, Severe acute respiratory syndrome coronavirus 2
aaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaa

SEQ ID NO: 4 - DNA, 249 bp, Severe acute respiratory syndrome coronavirus 2
aaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaat

SEQ ID NO: 5 - DNA, 153 bp, Severe acute respiratory syndrome coronavirus 2
gctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaac

SEQ ID NO: 6 - DNA, 132 bp, Severe acute respiratory syndrome coronavirus 2
attggctactaccgaagagctaccagacgaattcgtggtggtgacggtaaaatgaaagatctcagtccaagatggtatttctactacctaggaactgggccagaagctggacttccctatggtgctaacaaa

SEQ ID NO: 7 - DNA, 104 bp, Severe acute respiratory syndrome coronavirus 2
caagccttaccgcagagacagaagaaacagcaaactgtgactcttcttcctgctgcagatttggatgatttctccaaacaattgcaacaatccatgagcagtgc

SEQ ID NO: 8 - DNA, 20 bp, Artificial Sequence
  S1 forward primer
ttgccacctttgctcacaga

SEQ ID NO: 9 - DNA, 20 bp, Artificial Sequence
  S1 reverse primer
caccaaaggtccaaccagaa

SEQ ID NO: 10 - DNA, 26 bp, Artificial Sequence
  S1 probe
acacttctgcactgttagcgggtaca

SEQ ID NO: 11 - DNA, 21 bp, Artificial Sequence
  N2 forward primer
ccgcagagacagaagaaacag

SEQ ID NO: 12 - DNA, 20 bp, Artificial Sequence
  N2 reverse primer
gcactgctcatggattgttg

SEQ ID NO: 13 - DNA, 27 bp, Artificial Sequence
  N2 probe
aactgtgactcttcttcctgctgcaga

SEQ ID NO: 14 - DNA, 23 bp, Artificial Sequence
  S2 forward primer
gcacaaaagtttaacggccttac

SEQ ID NO: 15 - DNA, 25 bp, Artificial Sequence
  S2 reverse primer
cgctaacagtgcagaagtgtattga

SEQ ID NO: 16 - DNA, 27 bp, Artificial Sequence
  S2 probe
tgccacctttgctcacagatgaaatga

SEQ ID NO: 17 - DNA, 23 bp, Artificial Sequence
  S-M30 forward primer
tgtcatgatggaaaagcacactt

SEQ ID NO: 18 - DNA, 22 bp, Artificial Sequence
  S-M30 reverse primer
accagtgtgtgccatttgaaac

SEQ ID NO: 19 - DNA, 17 bp, Artificial Sequence
  S-M30 probe
cctcgtgaaggtgtctt

SEQ ID NO: 20 - DNA, 25 bp, Artificial Sequence
  OR1ab-1 forward primer
ttgctgcagtcataacaagagaagt

SEQ ID NO: 21 - DNA, 25 bp, Artificial Sequence
  ORF1ab-1 reverse primer
ttgctgcagtcataacaagagaagt

SEQ ID NO: 22 - DNA, 26 bp, Artificial Sequence
  ORF1ab-1 probe
tttgcctggcacgatattacgcacaa

SEQ ID NO: 23 - DNA, 21 bp, Artificial Sequence
  ORF1ab-2 forward primer
gtggtgcatcgtgttgtctgt

SEQ ID NO: 24 - DNA, 25 bp, Artificial Sequence
  ORF1ab-2 reverse primer
agggtcattagcacaagttgtaggt

SEQ ID NO: 25 - DNA, 26 bp, Artificial Sequence
  ORF1ab-2 probe
ctgccgttgccacatagatcatccaa

SEQ ID NO: 26 - DNA, 20 bp, Artificial Sequence
  ORF1ab-M9 forward primer
tgacaaagcttgcccattga

SEQ ID NO: 27 - DNA, 21 bp, Artificial Sequence
  ORF1ab-M9 reverse primer
gcacgacaaaacccacttctc

SEQ ID NO: 28 - DNA, 16 bp, Artificial Sequence
  ORF1ab-M9 probe
tgctgcagtcataaca

SEQ ID NO: 29 - DNA, 22 bp, Artificial Sequence
  ORF1ab-M16 forward primer
gggatcagacataccacccaaa

SEQ ID NO: 30 - DNA, 20 bp, Artificial Sequence
  ORF1ab-M16 reverse primer
tttgcacaatgcagaatgca

SEQ ID NO: 31 - DNA, 21 bp, Artificial Sequence
  ORF1ab-M16 probe
tgtgttaactgtttggatgac

SEQ ID NO: 32 - DNA, 23 bp, Artificial Sequence
  ORF1ab-M22 forward primer
ccgcgaagaagctataagacatg

SEQ ID NO: 33 - DNA, 19 bp, Artificial Sequence
  ORF1ab-M22 reverse primer
gacacccctcgacatcgaa

SEQ ID NO: 34 - DNA, 15 bp, Artificial Sequence
  ORF1ab-M22 probe
acgtgcatggattgg

SEQ ID NO: 35 - DNA, 24 bp, Artificial Sequence
  M-1 forward primer
cattgcttctttcagactgtttgc

SEQ ID NO: 36 - DNA, 26 bp, Artificial Sequence
  M-1 reverse primer
gcacgttgagaagaatgttagtttct

SEQ ID NO: 37 - DNA, 25 bp, Artificial Sequence
  M-1 probe
cgtacgcgttccatgtggtcattca

SEQ ID NO: 38 - DNA, 18 bp, Artificial Sequence
  M-M35 forward primer
gcttcgcagcgtgtagca

SEQ ID NO: 39 - DNA, 21 bp, Artificial Sequence
  M-M35 reverse primer
tgccaatcctgtagcgactgt

SEQ ID NO: 40 - DNA, 17 bp, Artificial Sequence
  M-M35 probe
tgactcaggttttgctg

SEQ ID NO: 41 - DNA, 22 bp, Artificial Sequence
  N-M36 forward primer
accgaagagctaccagacgaat

SEQ ID NO: 42 - DNA, 25 bp, Artificial Sequence
  N-M36 reverse primer
ccatcttggactgagatctttcatt

SEQ ID NO: 43 - DNA, 16 bp, Artificial Sequence
  N-M36 probe
cgtggtggtgacggta

SEQ ID NO: 44 - DNA, 63 bp, Artificial Sequence
  IPC1 template
cgctaacgggcgattctataagaatgcacattgcgtcgattcataagatgtctcgaccgcatg

SEQ ID NO: 45 - DNA, 19 bp, Artificial Sequence
  IPC1 forward primer
cgctaacgggcgattctat

SEQ ID NO: 46 - DNA, 24 bp, Artificial Sequence
  IPC1 probe
aagaatgcacattgcgtcgattca

SEQ ID NO: 47 - DNA, 20 bp, Artificial Sequence
  IPC1 Reverse primer
catgcggtcgagacatctta

SEQ ID NO: 48 - DNA, 64 bp, Artificial Sequence
  IPC2 template
atcaaggaaatgtttcatgaccaagcgaaaggccgctctacggaatggatttacgttactgcct

SEQ ID NO: 49 - DNA, 22 bp, Artificial Sequence
  IPC2 forward primer
atcaaggaaatgtttcatgacc

SEQ ID NO: 50 - DNA, 20 bp, Artificial Sequence
  IPC2 probe
aagcgaaaggccgctctacg

SEQ ID NO: 51 - DNA, 22 bp, Artificial Sequence
  IPC2 reverse primer
aggcagtaacgtaaatccattc

SEQ ID NO: 52 - DNA, 66 bp, Artificial Sequence
  IPC3 template
ctgctttgatcaacctccaatacctcgtatcattgtgcacctgccggtgaccactcaacgatgtgg

SEQ ID NO: 53 - DNA, 23 bp, Artificial Sequence
  IPC3 forward primer
ctgctttgatcaacctccaatac

SEQ ID NO: 54 - DNA, 24 bp, Artificial Sequence
  IPC3 probe
ctcgtatcattgtgcacctgccgg

SEQ ID NO: 55 - DNA, 19 bp, Artificial Sequence
  IPC3 reverse primer
ccacatcgttgagtggtca

SEQ ID NO: 56 - DNA, 71 bp, Artificial Sequence
  IPC4 template
cgcatgcgcagggtatatttggacagtatcgaatggactctgatgaacctttacaccgatctagaaacggg

SEQ ID NO: 57 - DNA, 19 bp, Artificial Sequence
  IPC4 forward primer
cgcatgcgcagggtatatt

SEQ ID NO: 58 - DNA, 29 bp, Artificial Sequence
  IPC4 probe
tggacagtatcgaatggactctgatgaac

SEQ ID NO: 59 - DNA, 23 bp, Artificial Sequence
  IPC4 reverse primer
cccgtttctagatcggtgtaaag

SEQ ID NO: 60 - DNA, 72 bp, Artificial Sequence
  IPC5 template
gaggcgaagattatcgtgtgtgccccgttatggtcgagttcggtcagagcgtcattgcgagtagtcgtttgc

SEQ ID NO: 61 - DNA, 22 bp, Artificial Sequence
  IPC5 forward primer
gaggcgaagattatcgtgtgtg

SEQ ID NO: 62 - DNA, 24 bp, Artificial Sequence
  IPC5 probe
cccgttatggtcgagttcggtcag

SEQ ID NO: 63 - DNA, 21 bp, Artificial Sequence
  IPC5 reverse primer
gcaaacgactactcgcaatga

SEQ ID NO: 64 - DNA, 23 bp, Artificial Sequence
  Assay 1 forward primer
gatgcaaatctgcaaaggaaaga

SEQ ID NO: 65 - DNA, 24 bp, Artificial Sequence
  Assay 1 probe
taagagggccatatgacgtggcaa

SEQ ID NO: 66 - DNA, 20 bp, Artificial Sequence
  Assay 1 reverse primer
cccaaacagcaagcctagat

SEQ ID NO: 67 - DNA, 18 bp, Artificial Sequence
  Assay 2 forward primer
tggcggtgtttgcagatt

SEQ ID NO: 68 - DNA, 22 bp, Artificial Sequence
  Assay 2 probe
ttctgacctgaaggctctgcgc

SEQ ID NO: 69 - DNA, 22 bp, Artificial Sequence
  Assay 2 reverse primer
acaactgaatagccaaggtgag

SEQ ID NO: 70 - DNA, 20 bp, Artificial Sequence
  Assay 3 forward primer
tgtctcggatccatctcact

SEQ ID NO: 71 - DNA, 24 bp, Artificial Sequence
  Assay 3 probe
tgagagcaacttcttcaagggccc

SEQ ID NO: 72 - DNA, 23 bp, Artificial Sequence
  Assay 3 reverse primer
aaactgcaacaacatcatagagc

SEQ ID NO: 73 - DNA, 21 bp, Artificial Sequence
  Assay 4 forward primer
actttgccaattgtacaggga

SEQ ID NO: 74 - DNA, 24 bp, Artificial Sequence
  Assay 4 probe
tgtctcggatccatctcactgcaa

SEQ ID NO: 75 - DNA, 20 bp, Artificial Sequence
  Assay 4 reverse primer
gcccttgaagaagttgctct

SEQ ID NO: 76 - DNA, 21 bp, Artificial Sequence
  Assay 5 forward primer
tatctagtgctgcagaaaggc

SEQ ID NO: 77 - DNA, 24 bp, Artificial Sequence
  Assay 5 probe
agggccatatgacgtggcaaatct

SEQ ID NO: 78 - DNA, 20 bp, Artificial Sequence
  Assay 5 reverse primer
cttggcgtcactttcagaga